(19) United States
(12) Patent Application Publication
(10) Pub. No.: US 2008/0318892 A1
(43) Pub. Date: Dec. 25, 2008

(54) METHODS AND FORMULATIONS FOR PROTECTING CELLS, AND FOR TREATING DISEASES AND CONDITIONS BY OPTIMIZING THE INTRACELLULAR CONCENTRATION OF NAD

(76) Inventors: John Geoffrey Pickering, London (CA); Eric van der Veer, The Hague (NL); Nica Borradaile, London (CA)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/033,735

(22) Filed: Feb. 19, 2008

Related U.S. Application Data
(63) Continuation-in-part of application No. 10/577,789.
(60) Provisional application No. 60/890,227, filed on Feb. 16, 2007.

(51) Int. Cl. A61K 31/7084; A61K 31/7088; A61P 43/00

(52) U.S. Cl. 514/44

(57) ABSTRACT

Pharmaceutical and cosmetic formulations and methods for optimizing the intracellular concentrations of NAD are provided. The present methods and compounds relate to the use of PBEF, PRPP and various forms of nicotinamide, individually or in combination, for therapeutic, cyto-protective, cosmetic and anti-aging purposes. PBEF, PRPP and nicotinamide, individually or in combination, as administered according to the invention, increase the metabolic fitness, health and performance of the cell, and thereby increase the cell's level of health during its lifecycle. By way of the present formulations and methods, optimizing the intracellular concentration of NAD+ facilitates a balance among the numerous intracellular interactions of NAD+, and its related pathways, such that the health of the cell and its resistance to stress and trauma are increased. This increased robustness attendant to the invention also facilitates the delay of apoptosis.

### RELATED APPLICATIONS

This application is a continuation in part of application Ser. No. 10/577,789, filed Apr. 28, 2006, which claims priority provisional patent application Ser. Nos. 60/515,648, filed Oct. 31, 2003, 60/573,339, filed May 24, 2004, and 60/624,625 filed Oct. 25, 2004. This application also claims priority to provisional patent application Ser. No. 60/890,227, filed Feb. 16, 2007. These applications are incorporated by reference in their entirety.

### FIELD OF THE INVENTION

The present invention relates generally to the protective and therapeutic uses of substances which optimise the intracellular concentration and availability of nicotinamide adenine dinucleotide (NAD⁺), and to related methods. NAD⁺-optimising substances include those such as pre-B cell colony-enhancing factor (PBEF, NamPT, Visfatin), 5-phosphoribosyl-pyrophosphate (PRPP), and SIRT1. The invention relates also to therapeutic and pharmaceutical formulations containing NAD-optimising compounds such as PBEF and PRPP, alone and in various combinations with other compounds such as nicotinamide, and/or SIRT1.

### BACKGROUND OF THE INVENTION

Current techniques used to enhance cyto-protection or to inhibit aberrant cell functions tend to work by reducing environmental and metabolic factors that can harm or poison cells and tissues. Unfortunately, many of these techniques are limited to the dynamics of the scavenging of free radicals, and protective coatings.

Animal cells, such as human cells, experience stress many times throughout their lifecycles often causing injury, death or irreparable DNA damage. The source of the stress can be environmental, such as radiation, toxic substances, and physical factors experienced by the cell such as mechanical injury due to trauma, and exposure to extreme weather. Other stresses include those caused by sunlight, dehydration and exposure to caustic or otherwise harsh chemicals. Other sources of stress can occur as a product of the biological system itself such as metabolic stress associated with diabetes, or during the natural phases of the cell cycle such as during times of proliferation and differentiation, and/or to the dynamics of carcinogenesis.

Age is the greatest risk factor for myocardial infarctions and strokes. This risk is partly attributable to an age-related decline in the ability of vascular cells to resist stress and effectively remodel the arterial wall. Vascular smooth muscle cells (SMCs) and vascular endothelial cells are especially important in this regard—the efficiency with which SMCs stabilize a developing atherosclerotic lesion determines whether the lesion will rupture, a potentially fatal event.

Revollo et al. postulate the regulatory interactions of Nampt, Nmnat and Sir2 (SIRT1) on the intracellular dynamics of NAD. These interactions occur after the synthesis of NAD. The NAD Biosynthetic Pathway Mediated by Nicotinamide Phosphoribosy-ltransferase Regulates Sir2 Activity in Mammalian Cells, The Journal of Biological Chemistry, Sep. 20, 2004. INSERT re Amgen/Samal PBEF; Song et al PBEF Enhancing Factor; and Hasmann et al, FK86.

Until the present compounds and methods, the healing, pharmaceutical, cosmetic and metabolic arts have been lacking in effective methods and formulations to improve the metabolic fitness of cells. By improving cellular metabolic fitness, cells are best prepared to experience such commonly occurring stress without incurring damage that would prematurely shorten the life of the cell, cause the cell to function improperly or degrade the physical appearance of the cell.

This raised the possibility that PBEF was involved in the synthesis of NAD. NAD is well known for its role in regulating the redox state of the cell. However, recent work has identified a number of other important NAD-dependent reactions, including histone deacetylation. Unlike the redox system, these newly discovered reactions deplete the pool of cellular NAD, and sometimes contribute to harmful imbalances in the cell. Imbalances in the state of NAD and its metabolic intermediates is well recognized as an indicator of disease states including but not limited to cardiovascular disease and cancer.

PBEF is also known as nicotinamide phosphoribosyltransferase (“Nampt”) and Visfatin and the terms are used interchangeably herein. PBEF is the rate-limiting enzyme for NAD⁺ biosynthesis from nicotinamide. The intracellular levels of NAD⁺ and nicotinamide are important for certain cell survival reactions, including those linked to the sirtuin family of protein deacetylases. Sirtuins, such as Sir2 and its mammalian homolog SIRT1, SIRT2 and SIRT3 consume NAD⁺ and generate nicotinamide as they hydrolytically remove a targeted acetyl group. Nicotinamide is a known inhibitor of NAD⁺-dependent deacetylation reactions. Therefore, pathways that both replenish NAD⁺ and clear nicotinamide could be vital to SIRT1, SIRT2 and SIRT3 activity.

The present inventors have discovered that optimizing the intracellular concentration of NAD⁺ facilitates a balance among the numerous intracellular interactions of NAD⁺ and its related pathways such that the health of the cell and its resistance to stress are increased. This increased robustness attendant to the invention also relates to a consequent delay of death of the cell. Moreover, the present inventors have discovered that Nampt is a longevity gene that extends the lifespan of human SMCs by activating SIRT1 and restraining the accumulation of p53. They also identify that the synergistic modulation of NamPT and SIRT1 is the basis to supporting extensive longevity. Similarly, the synergy between NamPT and SIRT1 provides the balance in the NAD pathway to support cell survival in response cell stress such as might be found in cardiovascular disease, and specifically in the example of glucose stress.

### SUMMARY OF THE INVENTION

The present invention is based on the unexpected discovery that PBEF and PRPP, alone or in combination with one another, or in combination with one or more forms of nicotinamide, or SIRT1, increase cell fitness, protect the cell against damage from stress factors, and increase the longevity of the cell.

Other aspects and features of the present invention will become apparent to those of ordinary skill in the art upon review of the following description of specific embodiments of the invention in conjunction with the accompanying Figures and claims. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The figures provided herein illustrate embodiments of the present invention, by way of example only, and not in a limiting way.

FIG. 1(A) shows Hoffman-modulated contrast images of HITB5 smooth muscle cells in M199 supplemented with 10% FBS (left images) and 6 days after culturing SMC's in serum-free M100 (right image);

FIG. 1(B) is a Northern Blot showing upregulation of the 3 major transcripts of PBEF in HITB5 SMC's following withdrawal of serum from cultures.

FIG. 1(C) is a Western Blot of cell lysates harvested from HITC6 SMC's before and after withdrawal of serum from cultures.

The images of FIG. 2 show the cellular elongation and aggregation of human Smooth Muscle Cells into multilayered ridges induced by the overexpression of PBEF. FIG. 2(A), shows Hoffman-modulated contrast images of sub-confluent (top panel) or post-confluent (middle panel) HITB5 Smooth Muscle Cells transduced with a retrovirus containing cDNA encoding EGFP alone (left image) or PBEF and EGFP from a bicistronic cassette (right image). The bottom panel of FIG. 2(A) depicts fluorescence images of the post-confluent SMC cultures, showing expression of the transgenes as indicated by EGFP fluorescence. Bar, 50 μm.

FIG. 2(B) shows the quantification of length-width ratios of control and PBEF-overexpressing Smooth Muscle Cells cultured in the presence of serum and 3 days after serum withdrawal.

FIG. 3 shows Western blots revealing expression of SMC differentiation markers in HITB5 SMC's infected with cDNA encoding EGFP alone (left image), and cDNA encoding both PBEF and EGFP (right image).

The images of FIG. 4 show the effect of PBEF of apoptosis. FIG. 4(A) shows cell accumulation over 11 days for control and PBEF-overexpressing HITB5 SMC's, cultured in M199 with 5% FBS.

FIG. 4(B) shows Thymidine incorporation into control and PBEF-overexpressing HITB5 SMC's, assessed by incubating cells in log-phase growth with 10 μCi/mL [³H]thymidine for 12 hours.

FIG. 4(C) presents fluorescence images of control and PBEF-overexpressing SMC's stained with Hoechst 33258 to identify nuclei (top panel), and for apoptotic nuclei by incubating with d-UTP fluorescein (bottom panel).

The images of FIG. 5 show the effect on SMC viability of the knockdown of PBEF expression and maturation induced by serum withdrawal. FIG. 5(A) shows Hoffman-modulated images of control HITC6 SMC's and HITC6-siRNA SMC's. Western blots showing PBEF protein expression for each cell line are shown.

FIG. 5(B) shows the length-width ratios of 50 randomly selected cells expressing either nsRNA 1248 or siRNA 1248.

FIG. 5(C) is a Western blot showing reduced expression of h-Caldesmon in HITC6-siRNA 1248 Smooth Muscle Cells.

FIG. 6 is a phylogenetic tree showing a tight evolutionary relationship between bacterial nicotinamide phosphoribosyltransferases and eukaryotic PBEF, including human PBEF.

The depictions of FIG. 7 show the effects of increasing PBEF levels on the levels of NAD⁺. FIG. 7(A) shows the HPLC analyses of deproteinized nucleotide extracts obtained from HEK293 cells transfected with PQCXIP or pQCXIP-PBEF, and HITC6 SMC's transduced with pQCXIP or pQCXIP-PBEF.

FIG. 7(B) shows quantitative data from 3 separate experiments for each cell type of FIG. 7(A), including Western blot insets depicting representative PBEF expression for control and for cells overexpressing PBEF.

FIG. 8 shows that increasing the levels of PBEF increases NAD⁺-dependent histone deacetylase activity in human Smooth Muscle Cells.

The images of FIG. 9 show the effect of overexpression of PBEF on the phenotype, vessel chimerism and investment of Smooth Muscle Cells. FIGS. 9(A) through 9(D) show sections of matrigel implants loaded with human SMC's. FIGS. 9(E) through 9(I) show sections stained with h-caldesmon and h-calponin. FIG. 9(J) illustrates the quantification of the proportion of microvessels invested by at least one EGFP-positive SMC.

FIG. 10 is a schematic diagram of the structure of PPRP;

FIG. 11 is a diagram of the structure of the essential layers and components of human skin;

FIGS. 12 A-C are phase contrast photomicrographs at 20× of vector-transduced or PBEF-overexpressing HITC6 SMC's in response to treatment with 5-phosphoribosyl-pyrophosphate (PRPP), demonstrating that the PRPP increases the health of smooth muscle cells compared to control cells;

FIGS. 13 A-B are phase contrast photomicrographs at 20× magnification of vector-transduced HITC6 SMC's in response to treatment with 5-phosphoribosyl-pyrophosphate (PRPP), demonstrating that the PRPP increases the health of smooth muscle cells compared to control cells;

FIGS. 14 A-B are phase contrast photomicrographs at 10× magnification of vector-transduced HITC6 SMC's in response to treatment with 5-phosphoribosyl-pyrophosphate (PRPP). SMC's were cultured in M199 1% FBS for 24 hours prior to addition of 500 uM PRPP.

FIG. 15 shows the salient biosynthetic pathways involved in the synthesis of NAD.

FIG. 16 shows the salient biosynthetic pathways involved in the utilization and regeneration of NAD.

FIG. 17] shows the decline of PBEF expression and Nampt enzyme activity in pre-senescent human SMCs.

FIG. 18 shows the regulation and extension of human SMC lifespan by Nampt.

FIG. 19 shows Nampt extending SMC lifespan and postponing senescence via enhanced SIRT1 activity and p53 degradation.

FIG. 20 shows the protection against NAD⁺-dependent oxidative stress-induced cell damage and p53 accumulation by Nampt.

FIG. 21 demonstrates that SIRT1 modestly extends human SMC lifespan. Cumulative population doublings of primary SMCs (FIG. 21A) and HITC6 SMCs (FIG. 21B) infected with retrovirus containing vector (pQCXIP) or vector with cDNA encoding human SIRT1. Graphs depict the averaged results from three longevity assessments and curves were fit using nonlinear regression.

FIG. 22 demonstrates that SMC senescence is heralded by an abrupt decline in the activities of both SIRT1 and Nampt. Western blots (top) show decreasing SIRT1 abundance and increasing p53 and p21 expression in HITC6 SMCs serially subcultured to senescence (S=subculture). SIRT1 deacetylase activity (middle) was determined in aliquots of the designated subculture by quantifying NAD⁺-dependent deacetylation of an acetylated peptide substrate in the presence of 1 μM TSA (*p<0.01 vs. S29, S31, and S33). Nampt enzyme activity (bottom) was assessed in the same subcultures by measuring [¹⁴C]nicotinamide mononucleotide production in lysates incubated with [¹⁴C]nicotinamide and phosphoribosylpyrophosphate (*p<0.001 vs. S29, S31, and S33).

FIG. 23 demonstrates that SIRT1 deacetylase activity in SMCs is dependent on Nampt. Graph depicting SIRT1 activity in HITC6 SMCs incubated with the Nampt antagonist FK866 (10 nM) or vehicle for 48 h. SIRT1 protein expression was unaffected (inset) (*p<0.005 vs. vehicle).

FIG. 24 shows a marked extension of human SMC lifespan by SIRT1 and Nampt. FIG. 24A shows a population doubling of HITC6 SMCs transduced with retrovirus containing control vectors, or cDNA encoding SIRT1, Nampt, or both SIRT1 and Nampt. The pronounced increase in replicative lifespan in SIRT1-Nampt SMCs corresponds to a substantial decrease in SMC senescence (FIG. 24B), determined after 35 days by staining for senescence-associated β-galactosidase activity at pH 6.0 (*p<0.05 vs. vector, tp<0.05 vs. SIRT1 and Nampt) (FIG. 24C) Graph showing amplification of SIRT1 activity by Nampt (*p<0.05 vs. vector, tp<0.001 vs. vector, SIRT1, and Nampt) (FIG. 24D). Graphs depicting abundance of p21 and p16^{INK4A} mRNA in human SMCs, assessed by quantitative realtime RT-PCR (*p<0.05 vs. vector, SIRT1, and Nampt).

FIG. 25 demonstrates the marked decrease in the levels (indicated by immunoblotting) and activity of Nampt indicated by decreased conversion of NAD⁺ to β-NMN−

FIG. 26 shows that, under normal glucose conditions, cells overexpressing Nampt demonstrate mildly improved longevity whereas under glucose stress conditions, cells overexpressing Nampt demonstrate significant augmented longevity. Sham transfected cells cultured under standard and high glucose conditions demonstrate typical patterns of longevity.

FIG. 27 shows that cell senescence is accompanied by typical aging and glucose stress-induced patterns of tetraploidy. In the condition of Nampt overexpression, the frequency of tetraploidy under age and glucose related stress is reduced to baseline levels.

FIG. 28 demonstrates that under conditions of glucose stress, vascular endothelial cells increase the endogenous levels of SIRT1.

It is an object of the invention to provide methods for protecting cells and tissues from harm by optimising the levels or concentrations of one or more forms of NAD in the cells and tissues.

It is a similar object of the invention to provide methods for repairing and healing cells and tissues from harm by optimising the levels or concentrations of one or more forms of NAD in the cells and tissues.

It is a further object of the invention to provide methods for increasing the longevity of cells by optimizing the level or concentration of NAD in the cells.

It is a further object of the invention to provide pharmaceutical formulations efficacious in optimising the levels or concentrations of one or more forms of NAD in the cells and tissues.

It is a further object of the invention to provide cosmetic formulations efficacious in optimising the levels or concentrations of one or more forms of NAD in the cells and tissues.

### DETAILED DESCRIPTION

The term “a cell” as used herein includes a single cell as well as a plurality or population of cells. Administering an agent to a cell includes both in vitro and in vivo administrations.

The term “effective amount” as used herein means an amount effective, at dosages and for periods of time necessary to achieve the desired result.

The term “animal” as used herein includes all members of the animal kingdom, including humans.

Pharmaceutical Compositions may be prepared using standard techniques known in the art.

In one embodiment, there is provided a method for treating a disease state characterized by cells with a non-optimal NAD cycle. The patient may be any animal, including a mammal, including a human.

“Treating” a condition or disease state refers to an approach for obtaining beneficial or desired results, including clinical results. Beneficial or desired clinical results can include, but are not limited to, alleviation or amelioration of one or more symptoms or conditions, diminishment of extent of disease, stabilization of the state of disease, prevention of development of disease, prevention of spread of disease, delay or slowing of disease progression, delay or slowing of disease onset, amelioration or palliation of the disease state, and remission (whether partial or total), whether detectable or undetectable. “Treating” can also mean prolonging survival of a patient beyond that expected in the absence of treatment. “Treating” can also mean inhibiting the progression of an aberrant condition, slowing the progression of injury, aging or malfunction temporarily, although more preferably, it involves halting the progression of the same permanently. As will be understood by a skilled person, results may not be beneficial or desirable if, the treatment results in adverse effects on the patient treated that outweigh any benefits effected by the treatment.

Through the work of the present inventors, the importance of maintaining an optimal balance in concentration of intracellular NAD+ to the robustness and completeness of cellular function is manifest in a number of ways, particularly when viewed in the context of the experiments reported herein. Moreover, the discovery of the present invention is unexpected, particularly in view of the related work of others.

In accordance with these and other objects, the invention provides formulations and methods for treating diseases or conditions in an animal. In one preferred embodiment, the method comprises the step of optimizing the intracellular activity of PBEF in the cells of at least one target tissue of the animal, wherein the optimizing of the activity of PBEF is effected by increasing the intracellular, or endogenous, concentration of the PBEF of the animal by a sufficient amount of PBEF.

In one preferred embodiment, optimization of the concentration of PBEF can be effected by administering to the animal a sufficient amount of PBEF to increase the intracellular concentration of the PBEF. The administration of the PBEF is preferably by at least one route, and the at least one route can be one or more of injection, oral administration, anal or other colonic administration, inhalation, intra-peritoneal administration, topical administration, intra-organ administration, infusion of a target tissue, transdermal and parenteral administration, including intravenous, intraperitoneal, subcutaneous, intramuscular, trans-epithelial, nasal, intrapulmonary, intrathecal, rectal and topical modes of administration or any other efficacious means. For example, in accordance with other objects of the invention, the optimization of PBEF levels can be performed by the methods of gene therapy, including the use of one or more viral vectors, such as adenoviruses, lentiviruses, adeno-associated viruses and non viral plasmid and cosmid vectors, and any other viral or prion vector amenable to optimizing the endogenous production and optimization of intracellular levels of PBEF.

Similarly, the present methods may be effected by promoting the endogenous production of PBEF in the cells of at least one target tissue of the animal, or in the whole animal, such as a human. Thus, promotion of intracellular production of PBEF can be effected, for example, by up-regulating the nucleic acid processes or mechanisms which support the production of PBEF, or by up-regulating the nucleic acid processes which increase the endogenous production of PBEF. Moreover, the present methods may be effected wherein the promotion of intracellular production of PBEF is effected by down-regulating the nucleic acid processes or mechanisms which repress the production of PBEF. Alternatively, the present methods may be practiced wherein the optimization of PBEF is effected by increasing the intracellular concentration of at least one modulator of PBEF, for example, by administering to the animal, such as a human, an effective amount of the modulator.

Administration of the modulator may be by any route known, and is preferably by at least one route, the at least one route being selected from routes such as injection, oral administration, anal or other colonic administration, inhalation, intra-peritoneal administration, topical administration, intra-organ administration, infusion of a target tissue, transdermal and parenteral administration, including intravenous, intraperitoneal, subcutaneous, intramuscular, trans-epithelial, nasal, intrapulmonary, intrathecal, rectal and topical modes of administration. In one preferred embodiment the modulator is PRPP.

In another preferred embodiment, the increase of PBEF is effected by promoting the endogenous production of PRPP in the cells of at least one target tissue of the animal, or in the whole animal, such as a human. The promotion of intracellular, or endogenous, production of PBEF may be effected, for example, by up-regulating the nucleic acid processes or mechanisms which increase the production of PRPP, or by down-regulating the nucleic acid processes or mechanisms which repress the production of PRPP.

In accordance with other methods of the invention, PRPP can be given in any efficacious form, or in combination with PBEF, or in combination with at least one form of nicotinamide, or in combination with PBEF and at least one form of nicotinamide. The nicotinamide may be in any efficacious form, such as in a substituted form, or in the form of one or more of nicotinic acid; nicotinic acid ribonucleotide; nicotinic acid ribonucleotide, reduced form; nicotinamide ribonucleotide; nicotinamide ribonucleotide, reduced form; nicotinamide riboside (and its reduced form); nicotinamide mononucleotide (NMN) and its reduced form; nicotinic acid adenine dinucleotide; nicotinic acid adenine dinucleotide, reduced form; nicotinamide adenine dinucleotide (NAD); nicotinamide adenine dinucleotide phosphate (NADP); nicotinamide adenine dinucleotide, reduced form (NADH); and nicotinamide adenine dinucleotide phosphate, reduced form (NADPH) and pharmaceutically acceptable salts thereof.

In accordance with other methods of the invention, PBEF activity may be modified by chemical or pharmaceutical compositions directly alone or in combination modifiers of SIRT1 activity. Modifiers of SIRT1 activity may encompass those factors dictating the intracellular levels of SIRT1 or those which affect the rate of its enzymatic function.

The present methods and formulations may be used to treat any disease or condition, and particularly those involving the disruption, harm or imbalance of the NAD pathways of cells including those diseases and conditions involving the vascular system including the heart, blood vessels and other portions of the cardiovascular system. Examples of such diseases and conditions include vascular insufficiency, vascular weakness, progeria, premature senescence of one or more tissues, aging, severe stress on one or more tissues, atherosclerosis, diabetes, arteriolesclerosis and re-vascularization of injured or weakened tissues or organs. The present methods and formulations may be used to treat any disease or condition which is a result of sever stress wherein the severe stress on one or more tissues is due to one or more of injury, malnutrition, disease, toxic shock and exposure.

Also in accordance with the present methods, the optimization of PBEF may be effected by increasing the intracellular concentration of at least one precursor of PBEF, for example, by administering to the animal an effective amount of the precursor.

Preferably, the administration of the precursor is by at least one route, and the at least one route is one or more of injection, oral administration, anal or other colonic administration, inhalation, intra-peritoneal administration, topical administration, intra-organ administration, infusion of a target tissue, transdermal and parenteral administration, including intravenous, intraperitoneal, subcutaneous, intramuscular, trans-epithelial, nasal, intra-pulmonary, intrathecal, rectal and topical modes of administration.

In one aspect, the precursor may be at least one form of nicotinamide.

In another aspect, the nicotinamide may be substituted or in the form of one or more of nicotinic acid; nicotinic acid ribonucleotide; nicotinic acid ribonucleotide, reduced form; nicotinamide ribonucleotide; nicotinamide ribonucleotide, reduced form; nicotinic acid adenine dinucleotide; nicotinic acid adenine dinucleotide, reduced form; nicotinamide adenine dinucleotide (NAD); nicotinamide adenine dinucleotide phosphate (NADP); nicotinamide adenine dinucleotide, reduced form (NADH); and nicotinamide adenine dinucleotide phosphate, reduced form (NADPH) and pharmaceutically acceptable salts thereof.

In accordance with still other advantages of the present invention, pharmaceutical and cosmetic formulations are provided. Formulations for optimizing the intracellular concentration of NAD of the invention include one or more of effective amounts of PRPP, PBEF, and nicotinamide with or without SIRT1. In compositions of the invention comprising nicotinamide, the nicotinamide may be substituted or in the form of one or more of nicotinic acid; nicotinic acid ribonucleotide; nicotinic acid ribonucleotide, reduced form; nicotinamide ribonucleotide; nicotinamide ribonucleotide, reduced form; nicotinic acid adenine dinucleotide; nicotinic acid adenine dinucleotide, reduced form; nicotinamide adenine dinucleotide (NAD); nicotinamide adenine dinucleotide phosphate (NADP); nicotinamide adenine dinucleotide, reduced form (NADH); and nicotinamide adenine dinucleotide phosphate, reduced form (NADPH) and pharmaceutically acceptable salts thereof.

A pharmaceutical or cosmetic composition of the invention may further comprise one or more of an effective amount of a pharmaceutically effective vehicle, a pharmaceutically effective diluent, a pharmaceutically effective cream, a pharmaceutically effective excipient, one or more pharmaceutically effective micelles, a pharmaceutically effective carrier, pharmaceutically acceptable concentrations of salt, buffering agents, preservatives and various compatible carriers. Preferably, compositions of the invention are adaptable for administration by at least one route, and the at least one route is one or more of injection, oral administration, anal or other colonic administration, inhalation, intra-peritoneal administration, topical administration, intra-organ administration, infusion of a target tissue, transdermal and parenteral administration, including intravenous, intraperitoneal, subcutaneous, intramuscular, trans-epithelial, nasal, intrapulmonary, intrathecal, rectal and topical modes of administration.

In accordance with still other objects of the invention, its compositions may be provided in the form of one or more of ingestible tablets, buccal tablets, troches, capsules, elixirs, suspensions, micelle encapsulations, syrups, wafers and the like, or enclosed or enclosable within hard or soft shell gelatin capsules. Moreover, the present compositions may further comprise one or more of an effective amount of a cosmetically effective vehicle, a cosmetically effective diluent, a cosmetically effective cream, a cosmetically effective excipient, one or more cosmetically effective micelles, a cosmetically effective carrier, cosmetically acceptable concentrations of salt, buffering agents, preservatives and various cosmetically compatible carriers.

As one of skill in the pharmaceutical or cosmetic arts will comprehend, numerous combinations and formulations of PBEF, and modulators of PBEF, and combinations of such factors as PRPP, nicotinamide, and SIRT1 and/or SIRT1 modulators are within the scope and spirit of the invention, as are numerous variations of the present methods.

Vascular smooth muscle cells (SMC's) can exist in an immature state with the capacity to proliferate and migrate (1). The switch from a proliferative/migratory SMC to a contractile SMC is referred to as SMC maturation and is a process that is central to vascular development, stability, and physiologic function (2). Maturation of SMC's is necessary to stabilize newly formed blood vessels and confer vasomotor reactivity (3). Similarly, replicating SMC's in injured arteries must eventually mature to a quiescent phenotype to terminate the remodeling process.

The primary function of vascular SMC's in their quiescent state is to contract and provide vascular tone (Owens, 1995) (Sobue, 1998) (Rybalkin, 2003). The healthy SMC, which morphologically is similar to HITB5 or HITC6 SMC's that have been cultured in serum-free media for prolonged periods (more than 72 hours) is characterized by an elongated appearance, increased expression of SMC contractile proteins, such as h-caldesmon, metavinculin, smooth muscle myosin heavy chain and calponin and a marked decrease in apoptosis (Li, Circ. Res, 1999). We have found that HITB5 and HITC6 SMC's that are in this state have a significant increase in their transcript and protein levels of PBEF, suggesting that PBEF is a novel factor involved in regulating SMC differentiation and maturation directly or supporting their survival through the differentiation and maturation process.

The molecular basis by which an immature SMC shifts to a functionally contractile cell is incompletely defined (4). This is partly due to a paucity of culture systems that recapitulate this critical, late phase of the SMC developmental program. Recently however, we cloned three adult vascular SMC lines that, in contrast to other human SMC preparations, could reversibly convert between a spread, immature state when cultured in the presence of serum to a highly elongated, mature state after serum withdrawal (5, 6). Whereas cultured human SMC's often die upon serum withdrawal, these cells displayed decreased apoptosis, increased contractile protein expression, and the ability to contract in response to vasoactive agonists. This system therefore provided us with an opportunity to seek out factors that enable an activated adult SMC to return to a surviving metabolically quiescent cell. Accordingly, we undertook differential display PCR and high-density microarray analyses to identify genes that were differentially expressed as a homogenous population of human SMC's executed this key shift in phenotype.

These surveys identified pre-B cell colony-enhancing factor (PBEF) as being consistently upregulated as SMC's switched to the mature, quiescent state. PBEF is a 52-55 kD protein that has been proposed to be a cytokine (7). Reported actions in this regard include synergizing with other cytokines to stimulate the maturation of pre-B cells (7), stimulating the expression inflammatory cytokines in amniotic epithelial cells (8), and prolonging neutrophil survival (9). However, the contention that PBEF is a secreted cytokine is controversial. PBEF does not have a signal sequence for secretion and the presence of PBEF in culture media has been suggested to be a consequence of activation-induced cell death, rather than secretion by either a classical or alternative pathway (10, 11). Moreover, PBEF has sequence similarity with bacterial nadv, a protein that confers bacteria with the ability to grow in nicotinamide adenine dinucleotide (NAD⁺)-deficient conditions (12, 13). In keeping with this, Rongvaux and co-workers have shown that mouse PBEF functions within the cell as a nicotinamide phosphoribosyltransferase (10). This enzyme catalyzes the rate-limiting step in the salvage pathway for NAD⁺ biosynthesis, whereby nicotinamide that is generated during NAD⁺-consuming reactions is utilized to regenerate NAD⁺ (14, 15).

The experimental data reported here indicates that phenotype switching of human vascular SMC's is dependent on PBEF. Whereas SMC's deficient in PBEF were compromised in their ability to elongate and express SMC differentiation markers, genetic augmentation of PBEF expression promoted SMC survival and conversion to a mature phenotype. An increasingly supported argument may be made that the role of PBEF is this case is in support of the survival of SMCs in response to cellular stress. These actions were associated with an increase in steady state NAD⁺ levels and increased NAD⁺-dependent histone deacetylase activity. SMC's with augmented expression of PBEF manifested enhanced ability to associate with endothelial cells and wrap around nascent blood vessels in a human-mouse chimeric vascular development model. These findings establish PBEF as a novel, intracellular regulator of vascular SMC phenotype and implicate PBEF-mediated NAD⁺ flux as a driver of human SMC maturation via promoting cell survival.

Extending the Lifespan of Human Cells. In order to examine the mechanism of PBEF to support SMC state shift, the inventors examined the ability of PBEF to extending the lifespan of human cells by mediating survival through the aging process. Extending the productive lifespan of human cells could have major implications for diseases of aging, such as atherosclerosis. The inventors have identified a relationship between aging of human vascular SMCs and Nampt, the rate-limiting enzyme for NAD⁺ salvage from nicotinamide. Replicative senescence of SMCs was preceded by a marked decline in the expression and activity of Nampt. Furthermore, reducing Nampt activity with the antagonist FK866 induced premature senescence in SMCs, assessed by serial quantification of the proportion of cells with senescence-associated β-galactosidase activity. In contrast, introducing the Nampt gene into aging human SMCs delayed senescence and substantially lengthened cell lifespan, together with enhanced resistance to oxidative stress. Nampt-mediated SMC lifespan extension was associated with increased activity of the NAD⁺-dependent longevity enzyme SIRT1 and was abrogated in Nampt-overexpressing cells transduced with a dominant-negative form of SIRT1 (H363Y). Nampt overexpression also reduced the fraction of p53 that was acetylated on lysine 382, a target of SIRT1, suppressed an age-related increase in p53 expression, and increased the rate of p53 degradation. Moreover, add-back of p53 with recombinant adenovirus blocked the anti-aging effects of PBEF. These data indicate that Nampt is a longevity gene that can add stress-resistant life to human SMCs, by optimizing SIRT1-mediated p53 degradation.

Extension of Human Cell Lifespan Through Synergistic Modulation of Nampt and SIRT1 Activity. Having established a relationship between the activity of Nampt and SIRT1 in the survival of SMCs, the inventors investigated the ability of Nampt and SIRT1 to extend the survival of aging SMCs. SIRT1 activity decreases with aging of SMCs. While SIRT1 levels decline modestly, it is the innate decline of the activity of Nampt that supports the decreased activity of SIRT1 though the production of an imbalance in the NAD+ cycle. Furthermore, addition of Nampt and SIRT1 into the system synergistically significantly promotes the extension of SMC lifespan relative to the augmentation of the activity of either Nampt or SIRT1 alone.

Synergy of Nampt and SIRT1 elevation during glucose stress. The present inventors have demonstrated that a synergy of Nampt and SIRT1 elevation during glucose stress in senescing aortic endothelial cells supports longevity and genome stability. In human aortic endothelial cells, prolonged cell culture and subconfluent passage is a classic model of cell senescence.

Cell senescence is associated with a marked decrease in the levels and activity of Nampt. To investigate whether the natural decline in Nampt activity induced by aging might affect the longevity of cells over time and in response to glucose stress, human aortic endothelial cells were stably transfected either with a sham reporter construct or Nampt transgene and a reporter construct. The cells were passaged to induce senescence under conditions of standard glucose (5 mM glucose) or glucose stress conditions (30 mM glucose) to stimulate conditions related to normal cardiovascular aging and augmented diabetic or other hyperglycemia-related conditions.

Sham transfected cells cultured under standard and high glucose conditions demonstrated typical patterns of longevity (FIG. 26). Senescence is accompanied by typical aging and glucose stress induced patterns of tetraploidy (FIG. 27). Nampt-overexpressing cells demonstrated only baseline levels of age-induced tetraploidy indicating a protection from genomic instability associated with senescence. Under normal glucose conditions, cells overexpressing Nampt demonstrated mildly improved longevity (FIG. 26) whereas, under glucose stress conditions, cells overexpressing Nampt demonstrated significantly augmented longevity. Moreover, the Nampt overexpressed cells survived longer in high glucose compared with normal glucose (FIG. 27). This indicates that Nampt overexpression supports longevity in aortic endothelial cells under conditions of glucose stress but not necessarily under normal culture conditions.

Both sham transfected and overexpressing Nampt aging aortic cells express elevated levels of endogenous SIRT1 relative to those maintained under normal glucose conditions (data not shown). As Nampt overexpression enhances longevity in senescing aortic endothelial cells under high glucose stress, it may be argued that while both Nampt overexpression and SIRT1 overexpression individually increase longevity to some extent, it is the synergistic elevation of both Nampt and SIRT1 activity and/or levels that is required to have the greatest effect upon longevity. This data suggest that the protection of endothelial cells from glucose related stress requires a synergy of Nampt and SIRT1 activity that may be obtained by augmenting Nampt activity alone, and relying on the stress-related elevation of SIRT1, or through the balanced augmentation of both Nampt and SIRT1 levels and/or activity.

The mechanism by which PBEF exerts its pleiotropic effects is controversial. It has been suggested that PBEF functions as a secreted cytokine (Samal, 1994) (Jia, 2004), while it has also been reported to be involved in the salvage pathway of NAD+biosynthesis as a nicotinamide phosphoribosyltransferase (Rongvaux, 2002) (Martin, 2001). From the results shoen herein, we believe that PBEF is an intracellular protein that catalyzes the rate-limiting reaction of the salvage pathway. This pathway involves the conversion of nicotinamide (NAm) and 5-phosphoribosyl-pyrophosphate (PRPP) to nicontinamide mononucleotide (NMN) (Pilz, JBC, 1984) (Willis, Adv. Enzyme Reg., 1989). In resting human lymphocytes, the PRPP pools are very low (Snyder, JCI, 1976), and these cells were characterized by an impaired ability to generate ATP, inability to respond to mitogenic stimuli, while activation of these cells results in expansion of NAD+ and PRPP pools concomitant with the repair of DNA single-strand breaks (Johnstone, Eur. J. Biochem, 1984)(Berger, Exp. Cell Res., 1982)(Williams, Exp. Cell Res., 1985). Carson, D. A., Seto, S., and Wasson, D. B. Pyridine Nucleotide Cycling and Poly(ADP-Ribose) Synthesis in Resting Human Lymphocytes. Journal of Immunology. 138: 1904-1907, 1987. Willis, R. C., Nord, L. D., Fujitaki, J. M., and Robins, R. K. Potent and Specific Inhibitors of Mammalian Phosphoribosylpyrophosphate (PRPP) Synthetase. Advances in Enzyme Regulation. 28: 167-180, 1989.

PPRP and Nicotinamide are molecules critical for the regeneration of NAD in animal cells. NAD+ is a critical coenzyme involved in oxidation-reduction reactions in the cell. Cellular maintenance of NAD+ levels for redox reactions is likely tightly regulated due to its critical importance in energy generation. However, recent work has identified a number of other NAD+-dependent reactions that, unlike the redox system, deplete the free pool of intracellular NAD+. As such, regeneration of NAD+ can be achieved by de novo biosynthesis in primarily the liver or via the salvage pathway in the peripheral tissues (Magni, CMLS, 2004)(Bender, Brit. Jour. Nutr, 1988). The catabolism of NAD(P)+ in the liver results in the release of nicotinamide into the blood, which is utilized by peripheral tissues for the regeneration of NAD+ (Bender, 1988). Conversion of nicotinamide to nicotinic acid by nicotinamide deamidase is an important aspect of NAD+ generation in bacteria and yeast (Anderson, Nature, 2003) (Schuette, Am. J. Physiol, 1983). However, it is believed that nicotinamide deamidase is not expressed in mammals (Schuette, Am. J. Physiol, 1983)(Rongvaux, Bioessays, 2003). Therefore, conversion of nicotinamide and 5-phosphoribosyl pyrophosphate to NMN by PBEF is one critical step for the regeneration of NAD+ in peripheral tissues (Rongvaux, Eur. J. Biochem, 2002), such as SMC's. The marked upregulation of PBEF in differentiating SMC's demonstrates that the differentiation of SMC's is an NAD+ consuming process. Thus an argument may be made that optimization and balance of the NAD cycle can support cell survival in cases of cell stress or cellular aging.

The following experimental methods were employed to elucidate the data provided herein. Unnecessary details of the employed methods have been omitted for the sake of efficiency. Nonetheless, one of skill in the art will comprehend with certainty the significance and scope of the results reported herein.

### EXAMPLE 1

The primary function of vascular SMCs in their quiescent state is to contract and provide vascular tone (Owens, 1995) (Sobue, 1998) (Rybalkin, 2003). The healthy SMC, which morphologically is similar to HITB5 or HITC6 SMCs that have been cultured in serum-free media for prolonged periods (more than 72 hours) is characterized by an elongated appearance, increased expression of SMC contractile proteins, such as h-caldesmon, metavinculin, smooth muscle myosin heavy chain and calponin and a marked decrease in apoptosis (Li, Circ. Res, 1999). The inventors have found that HITB5 and HITC6 SMCs that are in this state have a significant increase in their transcript and protein levels of PBEF, demonstrating that PBEF is a novel factor involved in regulating SMC differentiation and maturation.

The Smooth Muscle Cell lines designated HITB5 and HITC6, in contrast to other vascular preparations, are capable of reversibly converting their phenotype between a spread, noncontractile state in the presence of serum and an elongated, contractile state after serum withdrawal (Li, Circ. Res, 1999). The withdrawal of serum induces in these cells a stress response, one that requires the cells to adapt to ensure survival. Primary cultures of SMCs cultured from explants are generally incapable of acquiring a truly, differentiated state in such stress conditions, and display an increased apoptotic rate. One of the hallmarks of HITB5 and HITC6 SMCs is their ability to adapt to media that is serum-free, suggesting that they possess the capability to activate a stress response that is sufficient to deal with the decreased abundance of nutrients and mitogens. Li, S., Sims, S., Jiao, Y., Chow, L. H., and Pickering, J. G. Evidence From a Novel Human Cell Clone That Adult Vascular Smooth Muscle Cells Can Convert Reversibly Between Noncontractile and Contractile Phenotypes. Circulation Research. 85(4): 338-348, 1999.

We show that vector-transduced or PBEF-overexpressing HITC6 SMCs in response to treatment with 5-phosphoribosyl-pyrophosphate (PRPP), demonstrating that the PRPP increases the health of cells compared to control cells;

The mechanism by which PBEF exerts its pleiotropic effects is controversial. It has been suggested that PBEF functions as a secreted cytokine (Samal, 1994) (Jia, 2004), while it has also been reported to be involved in the salvage pathway of NAD+ biosynthesis as a nicotinamide phosphoribosyltransferase (Rongvaux, 2002)(Martin, 2001). We believe that PBEF is an intracellular protein that catalyzes the rate-limiting reaction of the salvage pathway. This involves the conversion of nicotinamide (NAm) and 5-phosphoribosyl-pyrophosphate (PRPP) to nicontinamide mononucleotide (NMN) (Pilz, JBC, 1984) (Willis, Adv. Enzyme Reg., 1989). In resting human lymphocytes, the PRPP pools are very low (Snyder, JCI, 1976), and these cells were characterized by an impaired ability to generate ATP, inability to respond to mitogenic stimuli, while activation of these cells results in expansion of NAD+ and PRPP pools concomitant with the repair of DNA single-strand breaks (Johnstone, Eur. J. Biochem, 1984)(Berger, Exp. Cell Res., 1982)(Williams, Exp. Cell Res., 1985). Carson, D. A., Seto, S., and Wasson, D. B. Pyridine Nucleotide Cycling and Poly(ADP-Ribose) Synthesis in Resting Human Lymphocytes. Journal of Immunology. 138: 1904-1907, 1987. Willis, R. C., Nord, L. D., Fujitaki, J. M., and Robins, R. K. Potent and Specific Inhibitors of Mammalian Phosphoribosylpyrophosphate (PRPP) Synthetase. Advances in Enzyme Regulation. 28: 167-180, 1989.

PPRP and Nicotinamide are molecules critical for the regeneration of NAD in animal cells. NAD+ is a critical coenzyme involved in oxidation-reduction reactions in the cell. Cellular maintainance of NAD+ levels for redox reactions is likely tightly regulated due to its critical importance in energy generation. However, recent work has identified a number of other NAD+-dependent reactions that, unlike the redox system, deplete the free pool of intracellular NAD⁺. As such, regeneration of NAD+ can be achieved by de novo biosynthesis in primarily the liver or via the salvage pathway in the peripheral tissues (Magni, CMLS, 2004) (Bender, Brit. Jour. Nutr, 1988). The catabolism of NAD(P)+ in the liver results in the release of nicotinamide into the blood, which is utilized by peripheral tissues for the regeneration of NAD+ (Bender, 1988). Conversion of nicotinamide to nicotinic acid by nicotinamide deamidase is an important aspect of NAD+ generation in bacteria and yeast (Anderson, Nature, 2003) (Schuette, Am. J. Physiol, 1983). However, it is believed that nicotinamide deamidase is not expressed in mammals (Schuette, Am. J. Physiol, 1983) (Rongvaux, Bioessays, 2003). Therefore, conversion of nicotinamide and 5-phosphoribosyl pyrophosphate to NMN by PBEF is likely critical for the regeneration of NAD+ in peripheral tissues (Rongvaux, Eur. J. Biochem, 2002), such as SMCs. The marked upregulation of PBEF in differentiating SMCs demostrating that the SMC differentiation is a NAD+ consuming process.

Pre-B Cell Colony Enhancing Factor is Upregulated During SMC Maturation. SMC maturation entails the final stages of the SMC developmental program and confers cells with the capacity to contract. The generation of clonal populations of SMC's from the human internal thoracic artery, designated HITB5 and HITC6, which can convert from a proliferative state to a contractile SMC (5, 6), enabled us to screen for endogenous factor involved in this phenotype conversion.

The Smooth Muscle Cell lines designated HITB5 and HITC6, in contrast to other vascular preparations, are capable of reversibly converting their phenotype between a spread, noncontractile state in the presence of serum and an elongated, contractile state after serum withdrawal (Li, Circ. Res, 1999). The withdrawal of serum induces in these cells a stress response, one that requires the cells to adapt to ensure survival. Primary cultures of SMC's cultured from explants are generally incapable of acquiring a truly, differentiated state in such stress conditions, and display an increased apoptotic rate. One of the hallmarks of HITB5 and HITC6 SMC's is their ability to adapt to media that is serum-free, suggesting that they possess the capability to activate a stress response that is sufficient to deal with the decreased abundance of nutrients and mitogens. Li, S., Sims, S., Jiao, Y., Chow, L. H., and Pickering, J. G. Evidence From a Novel Human Cell Clone That Adult Vascular Smooth Muscle Cells Can Convert Reversibly Between Noncontractile and Contractile Phenotypes. Circulation Research. 85(4): 338-348, 1999.

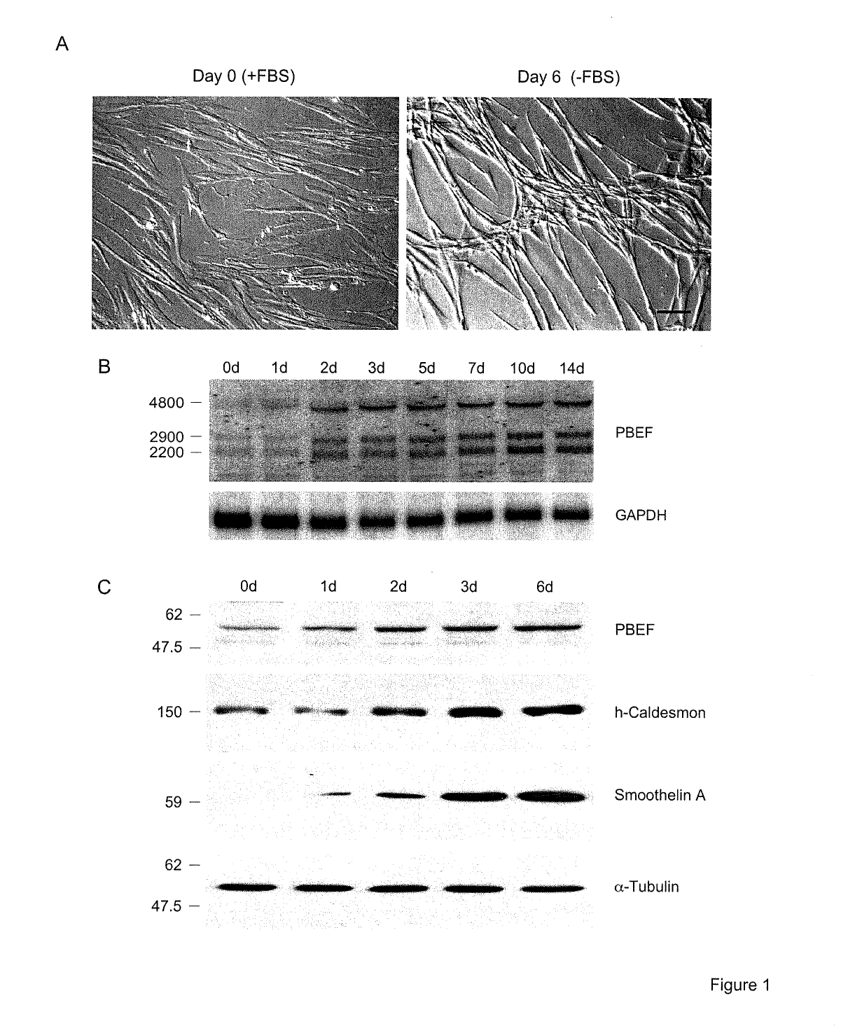

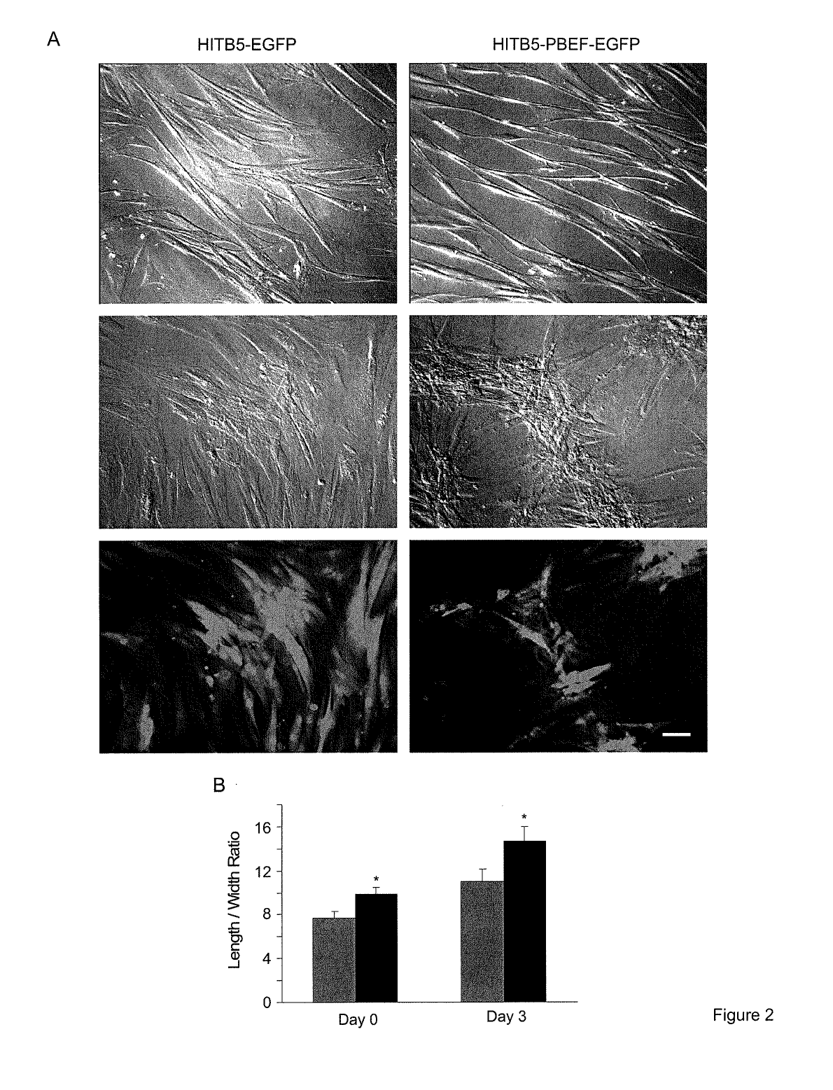

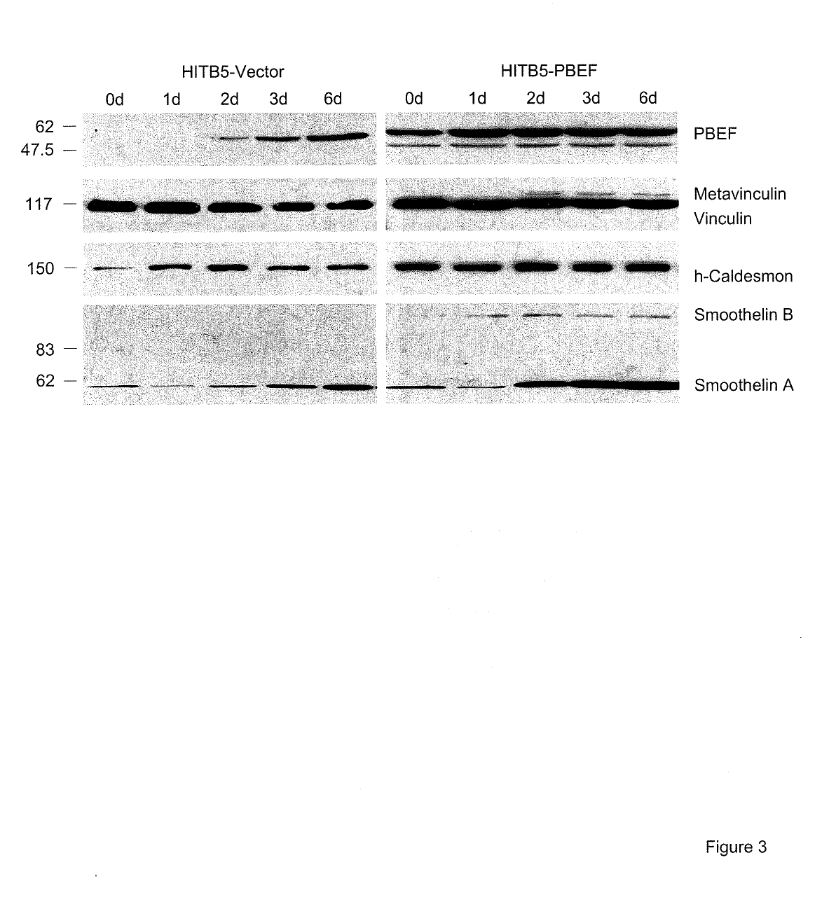

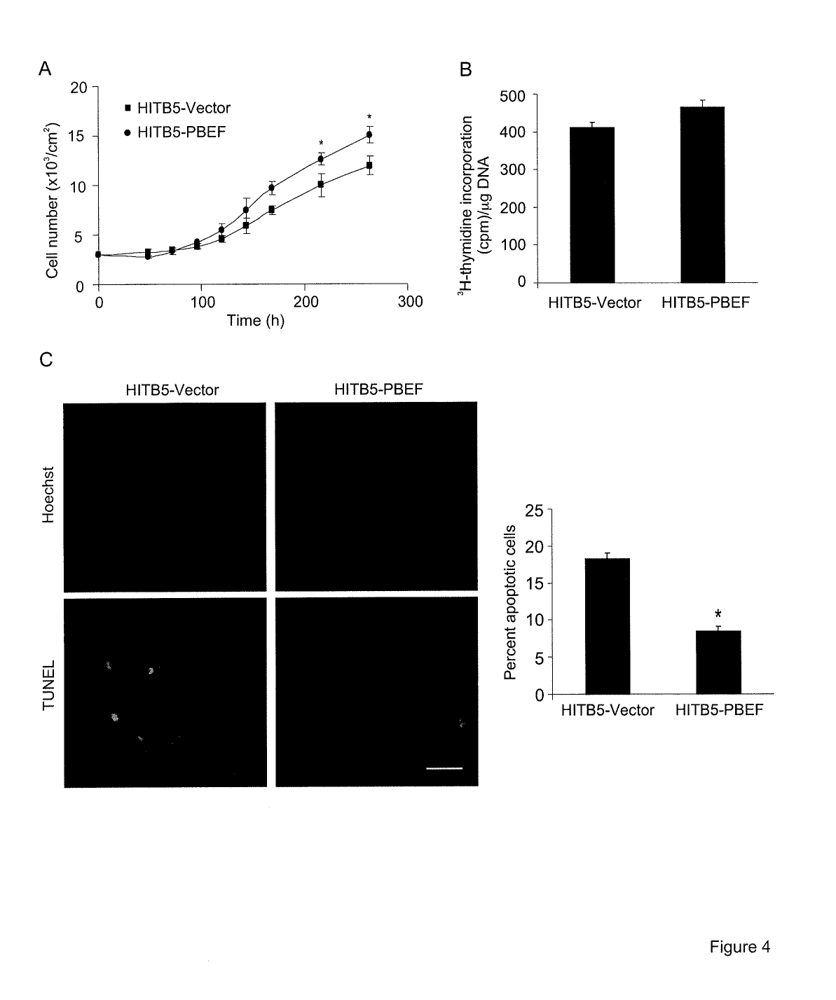

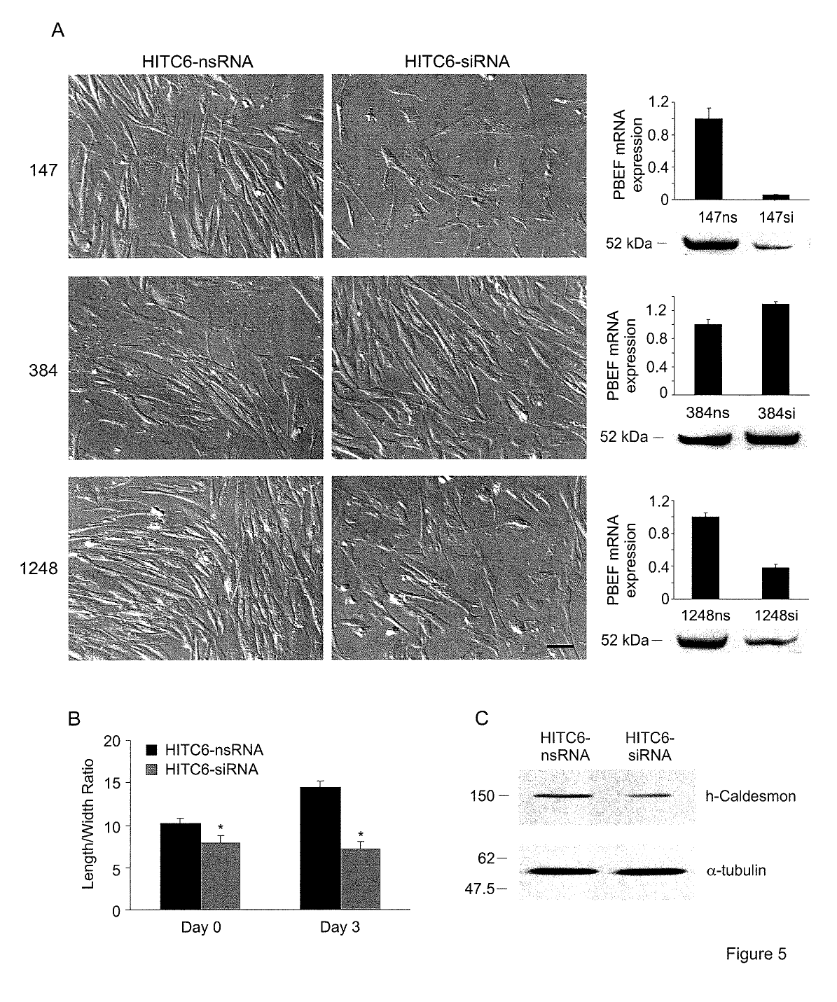

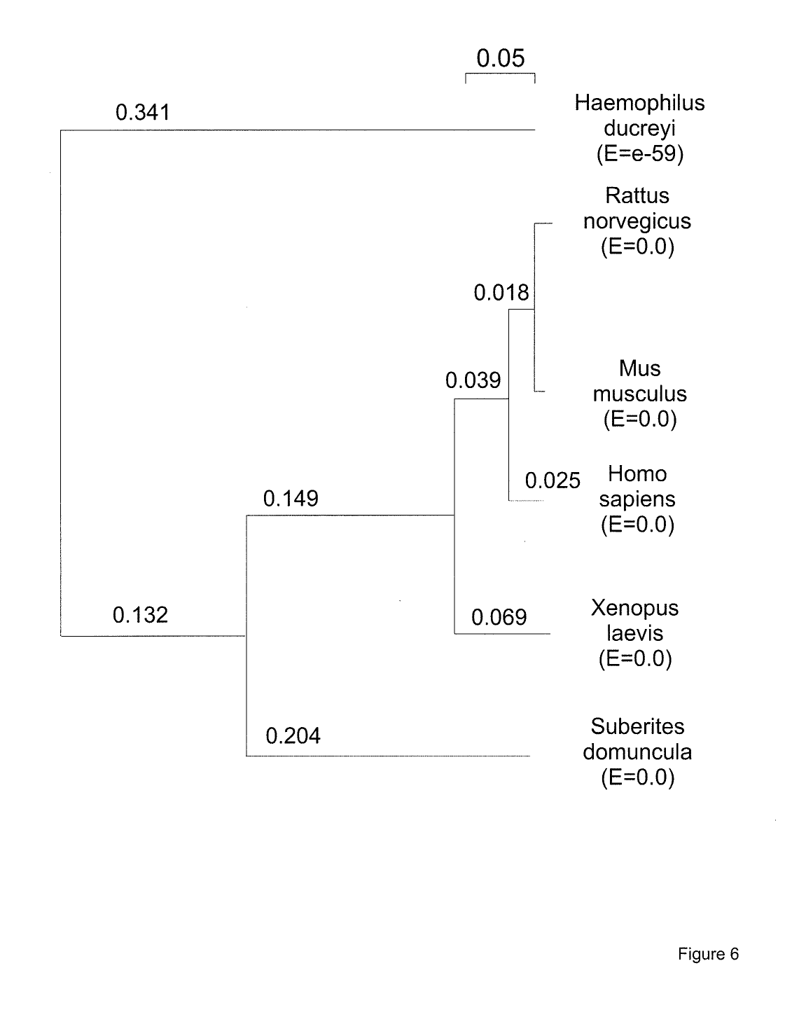

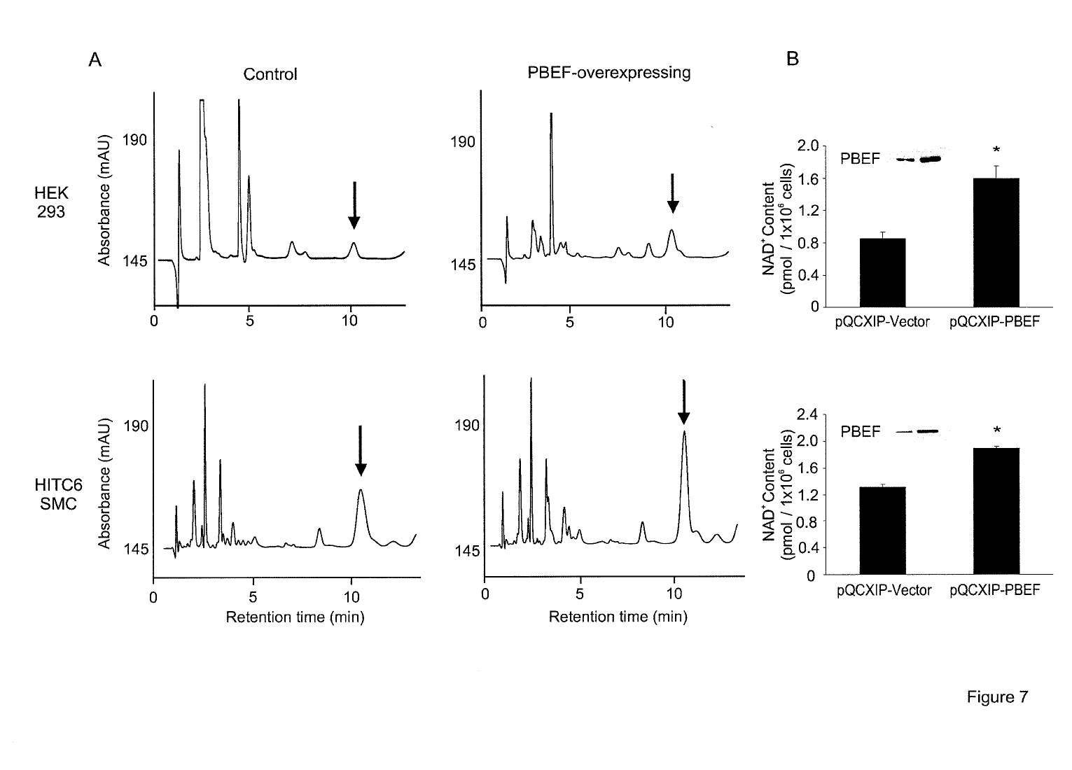

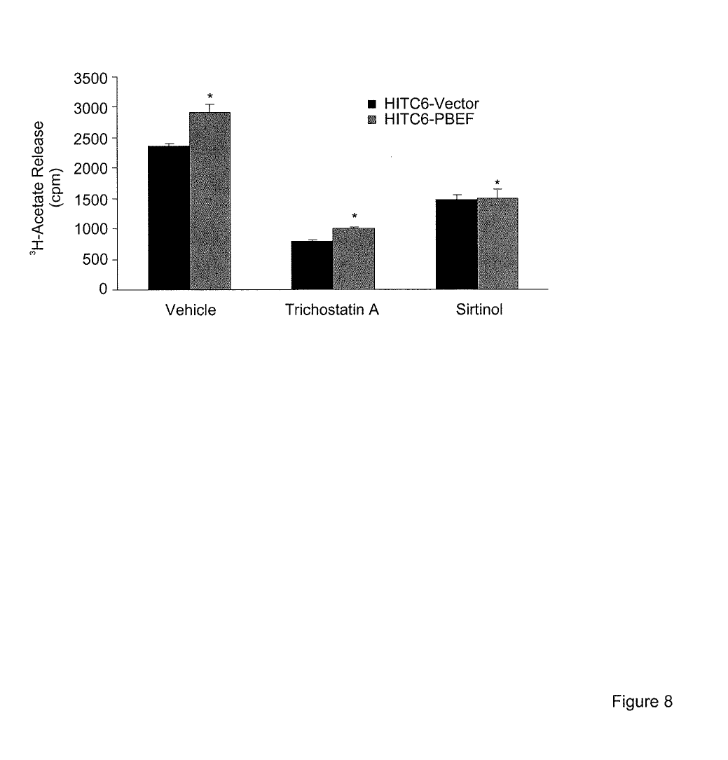

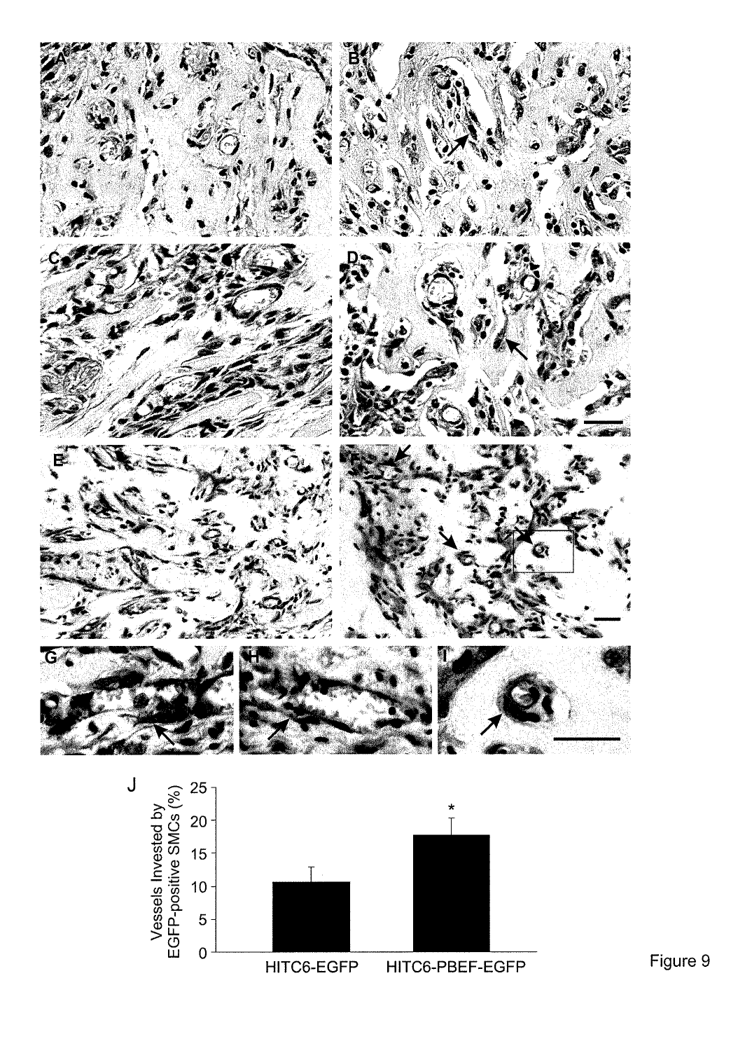

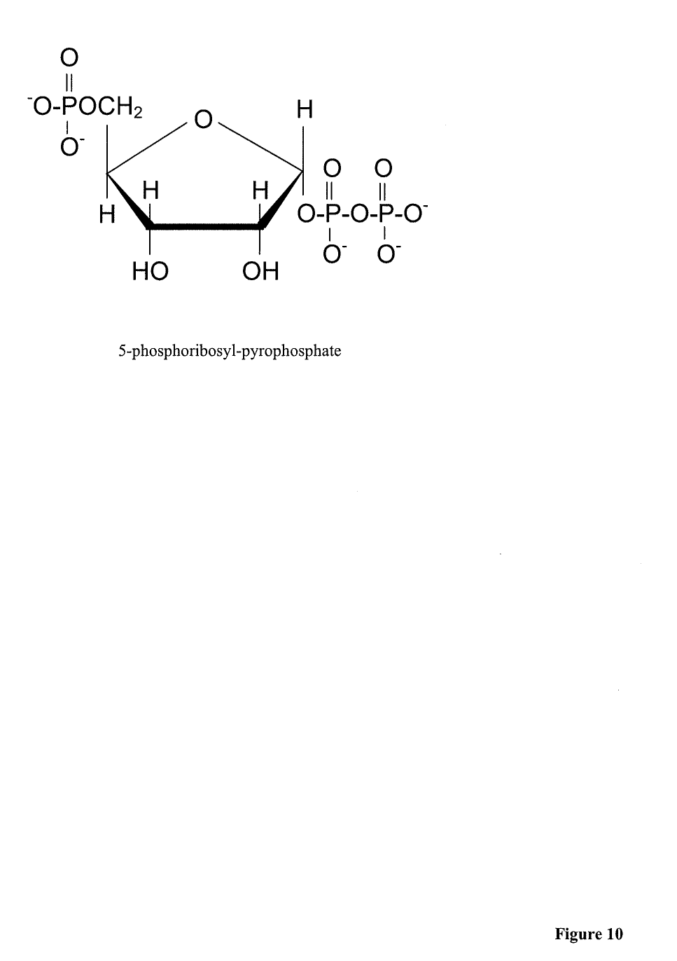

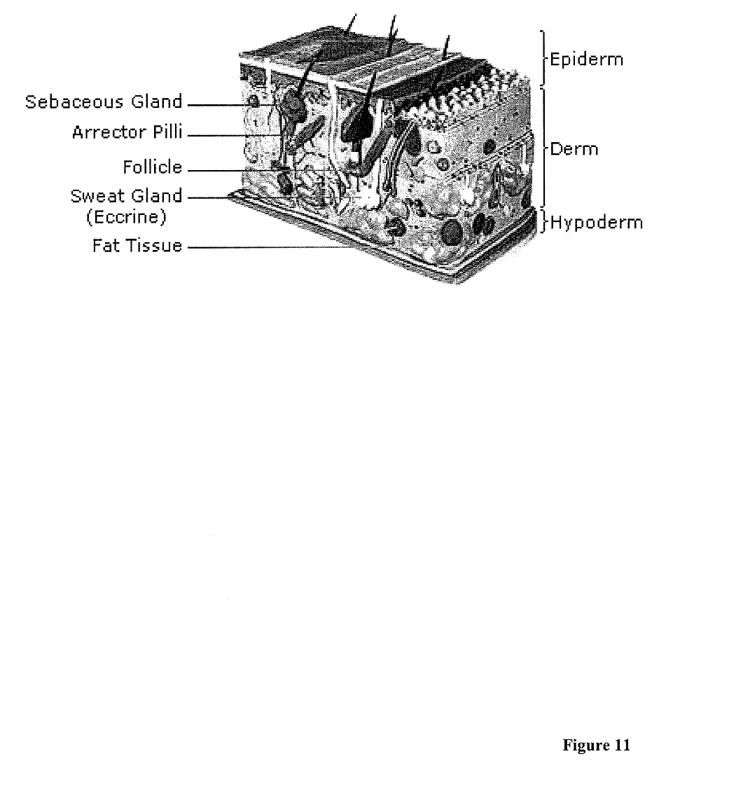

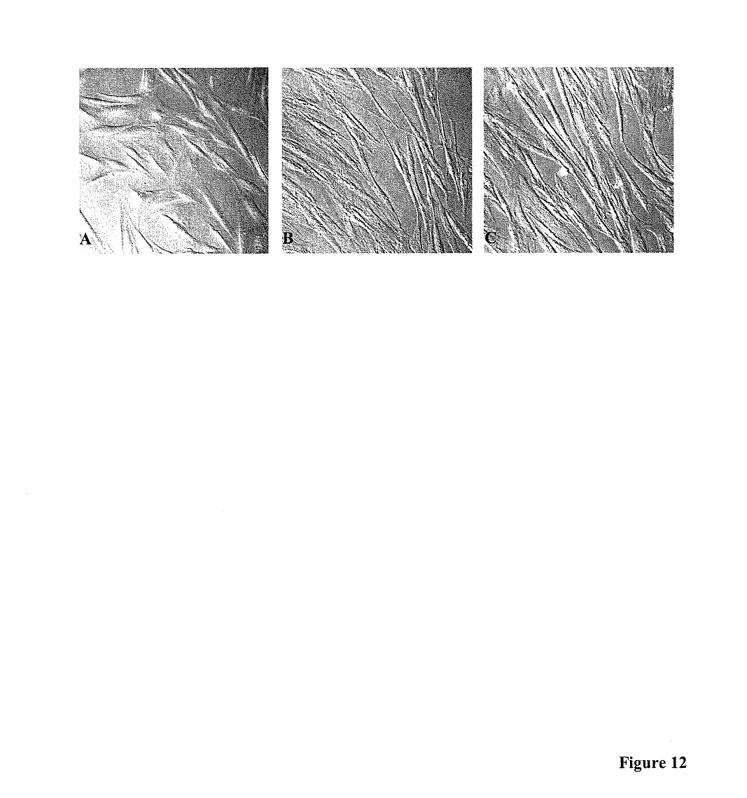

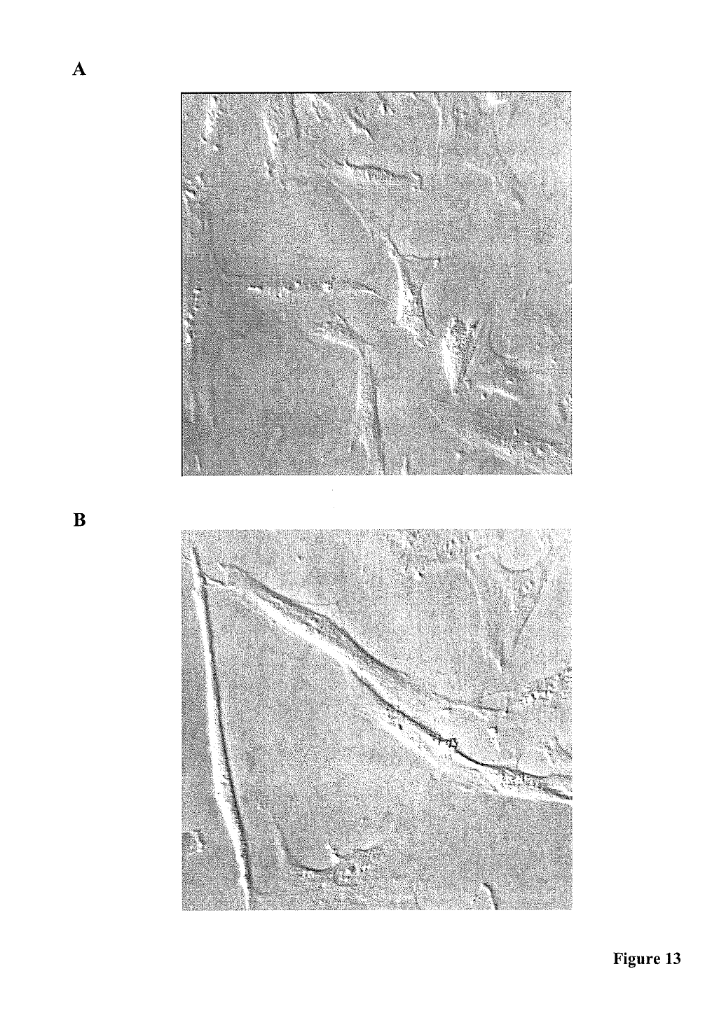

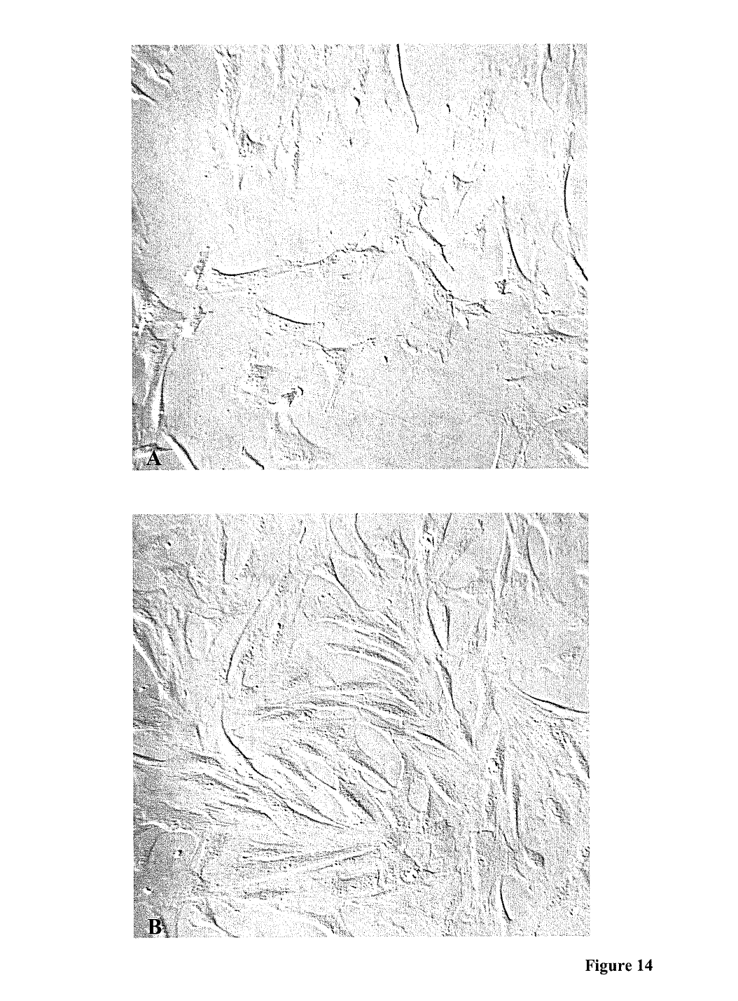

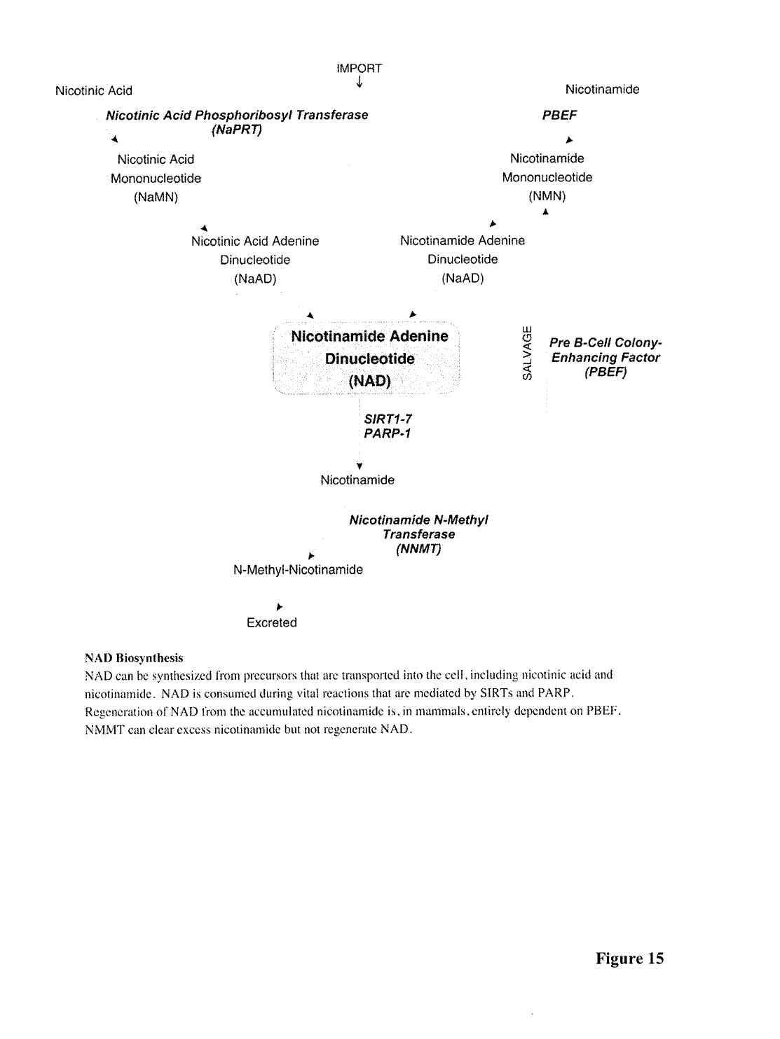

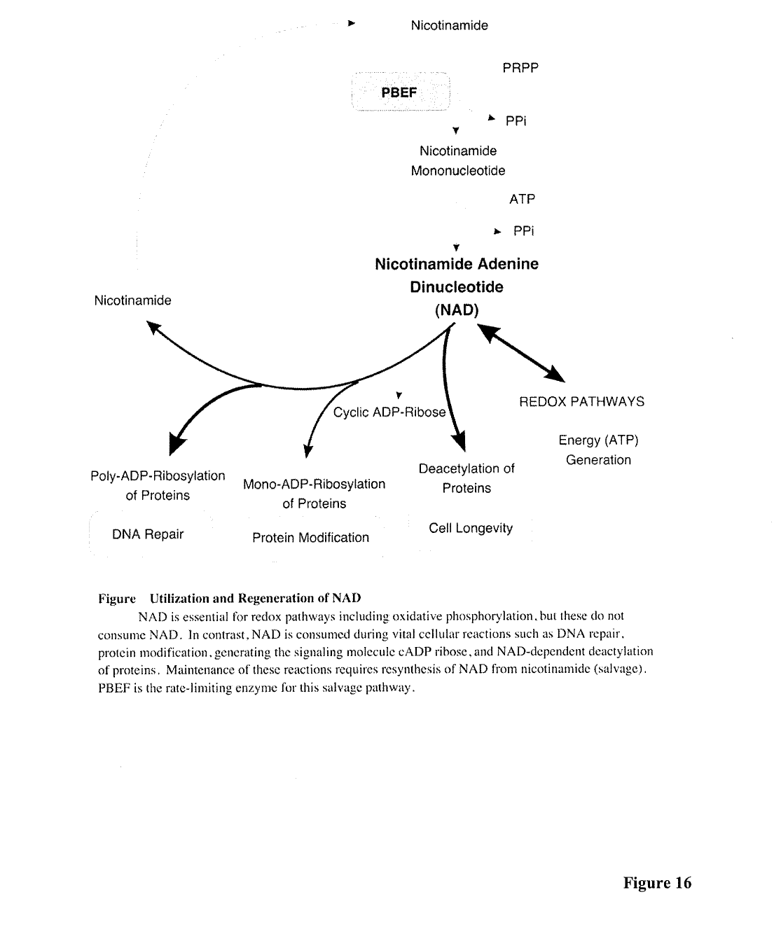

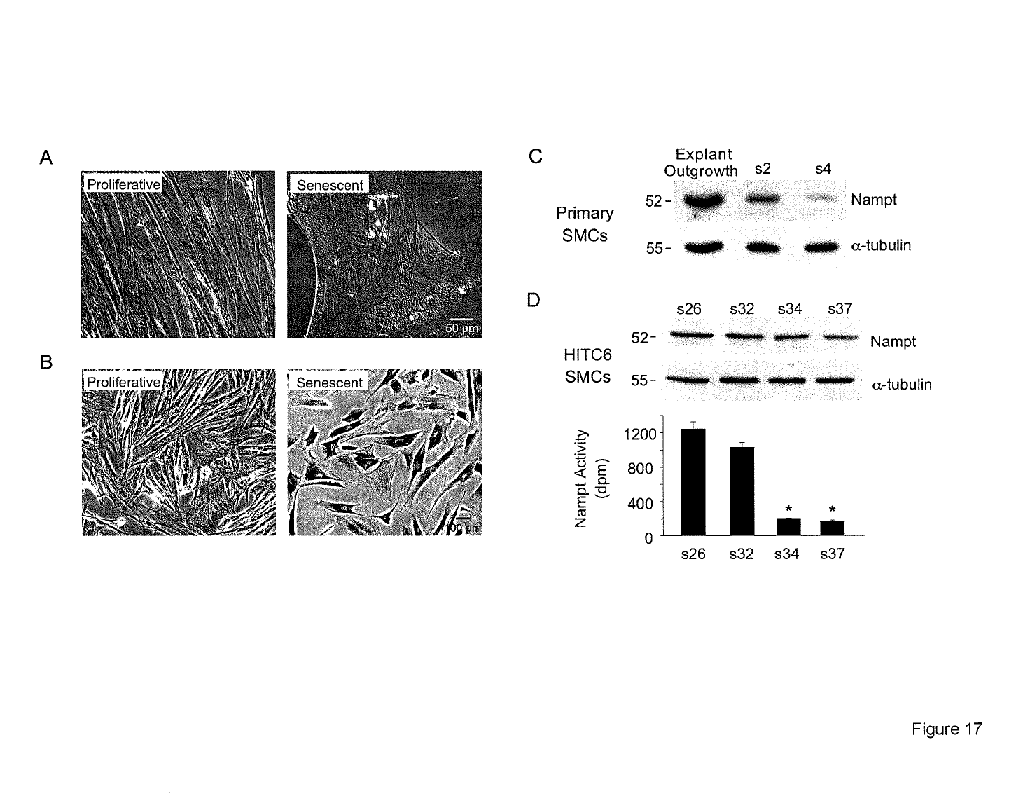

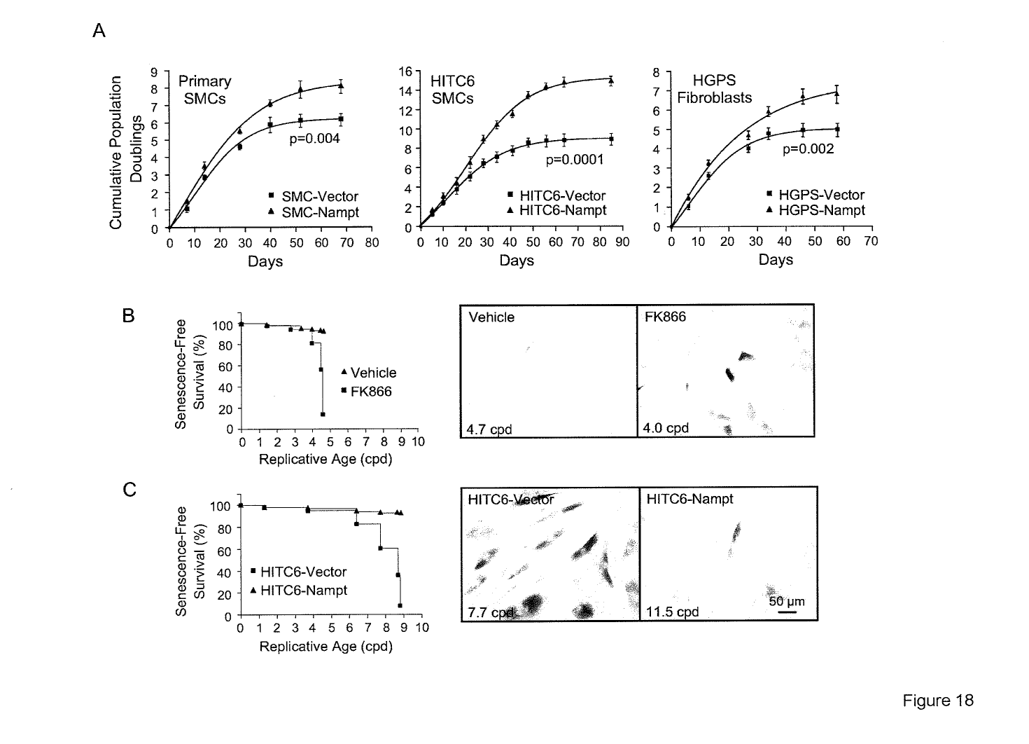

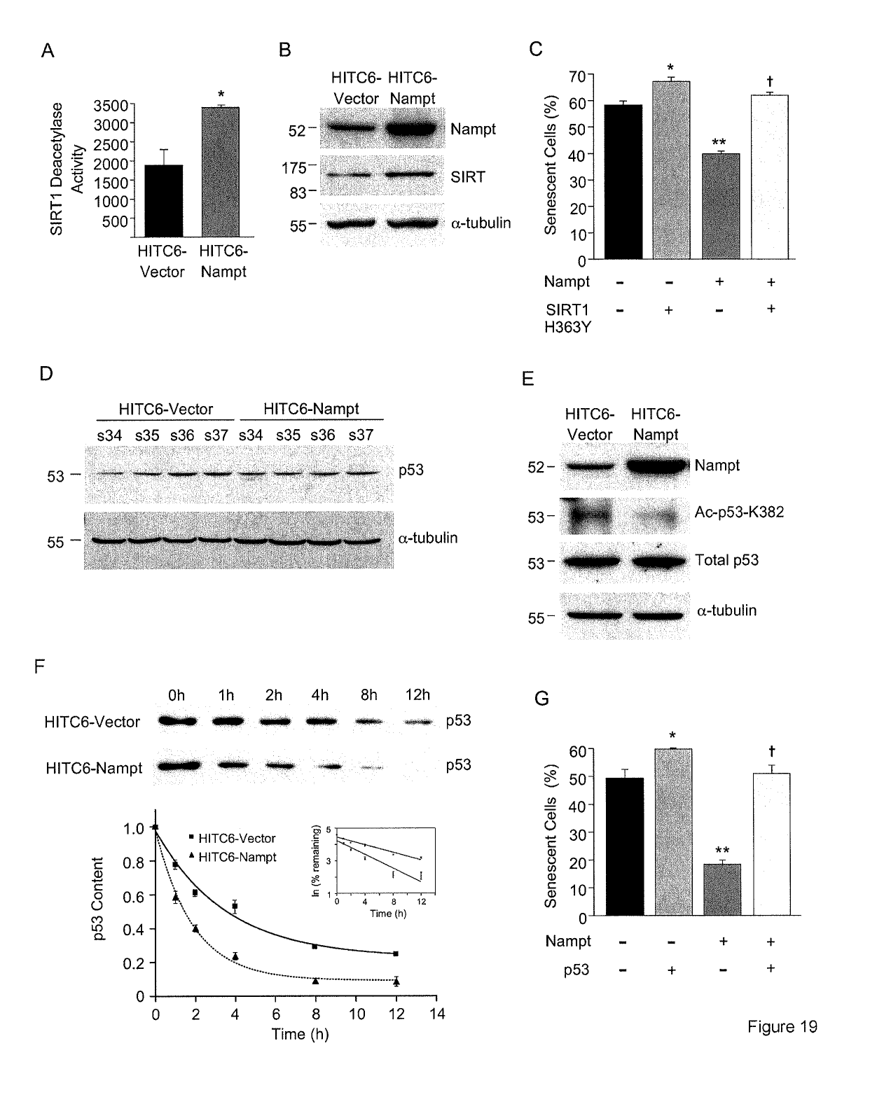

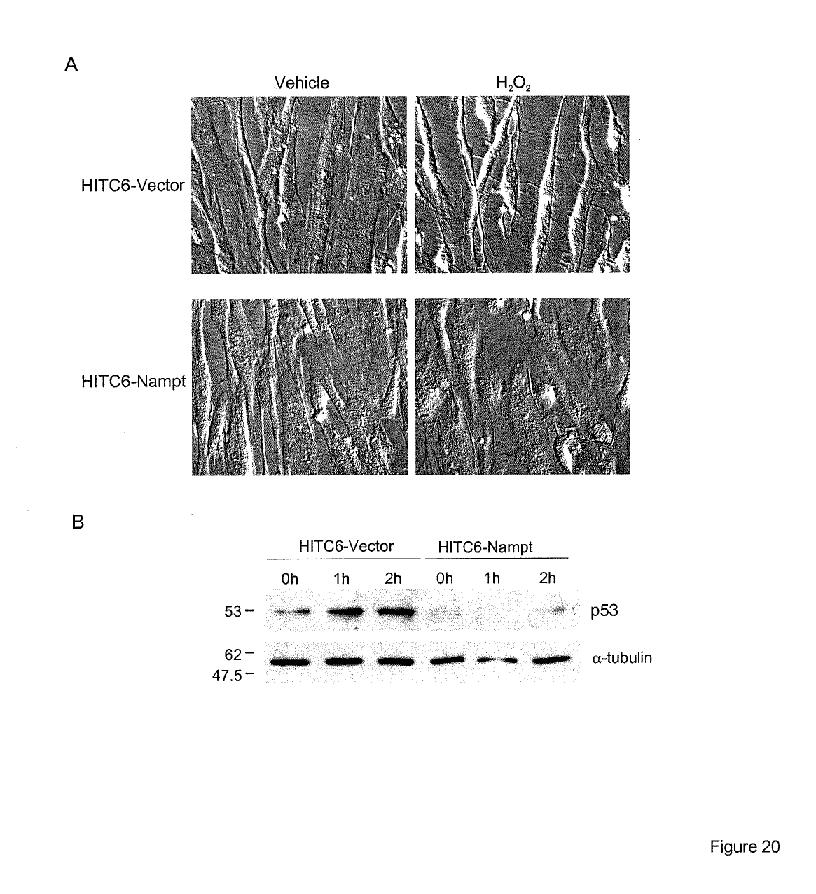

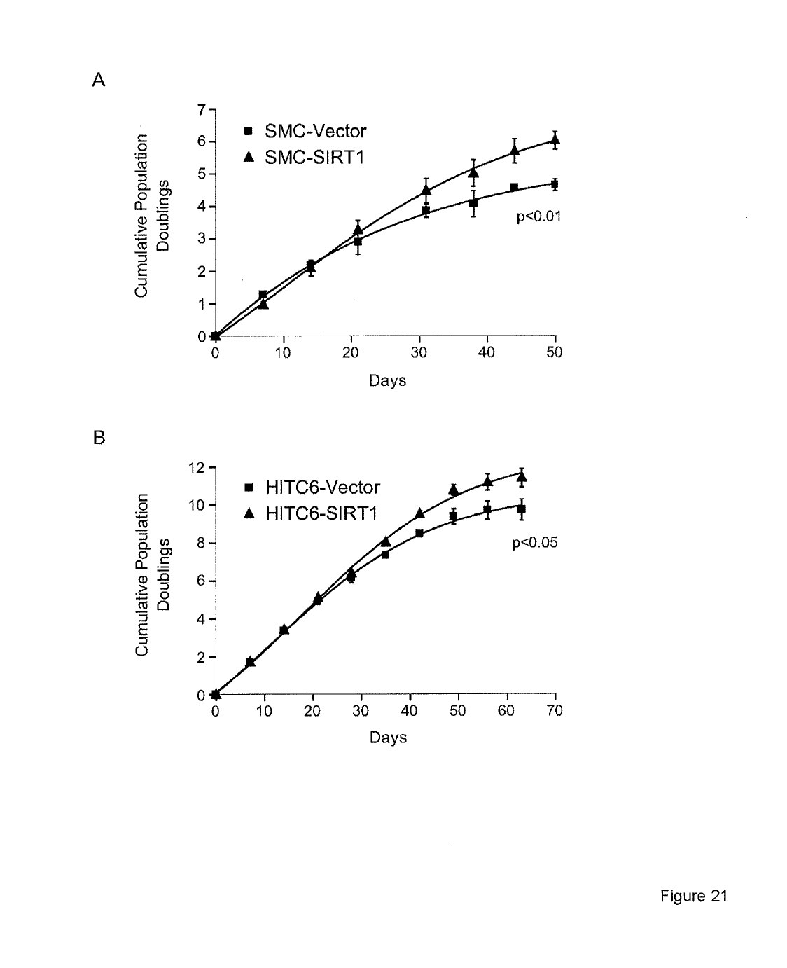

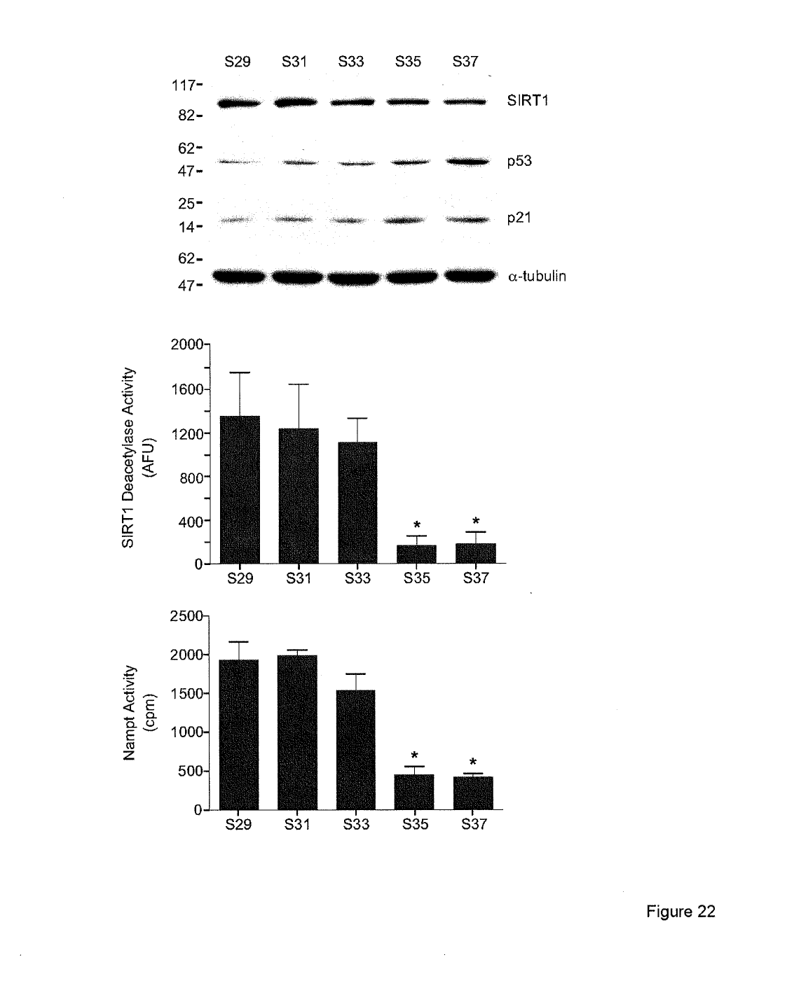

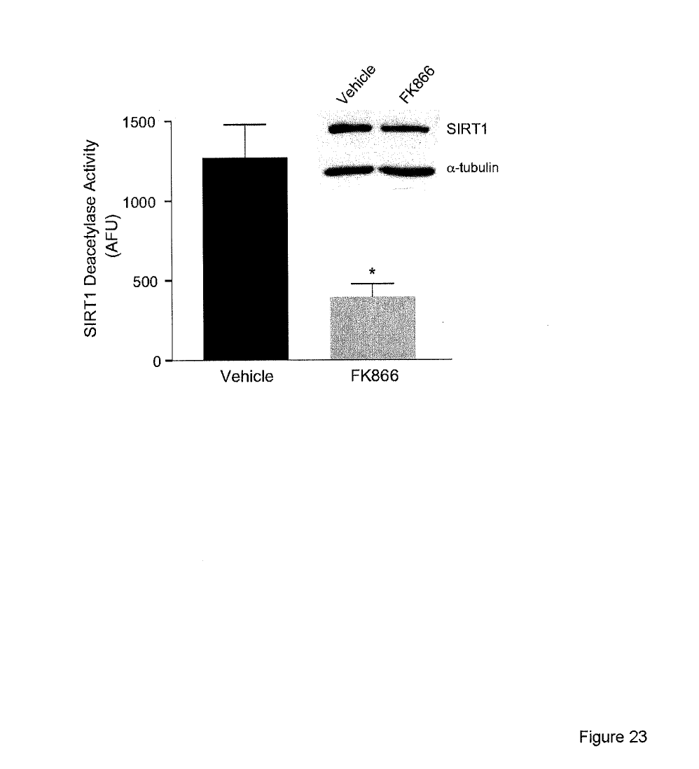

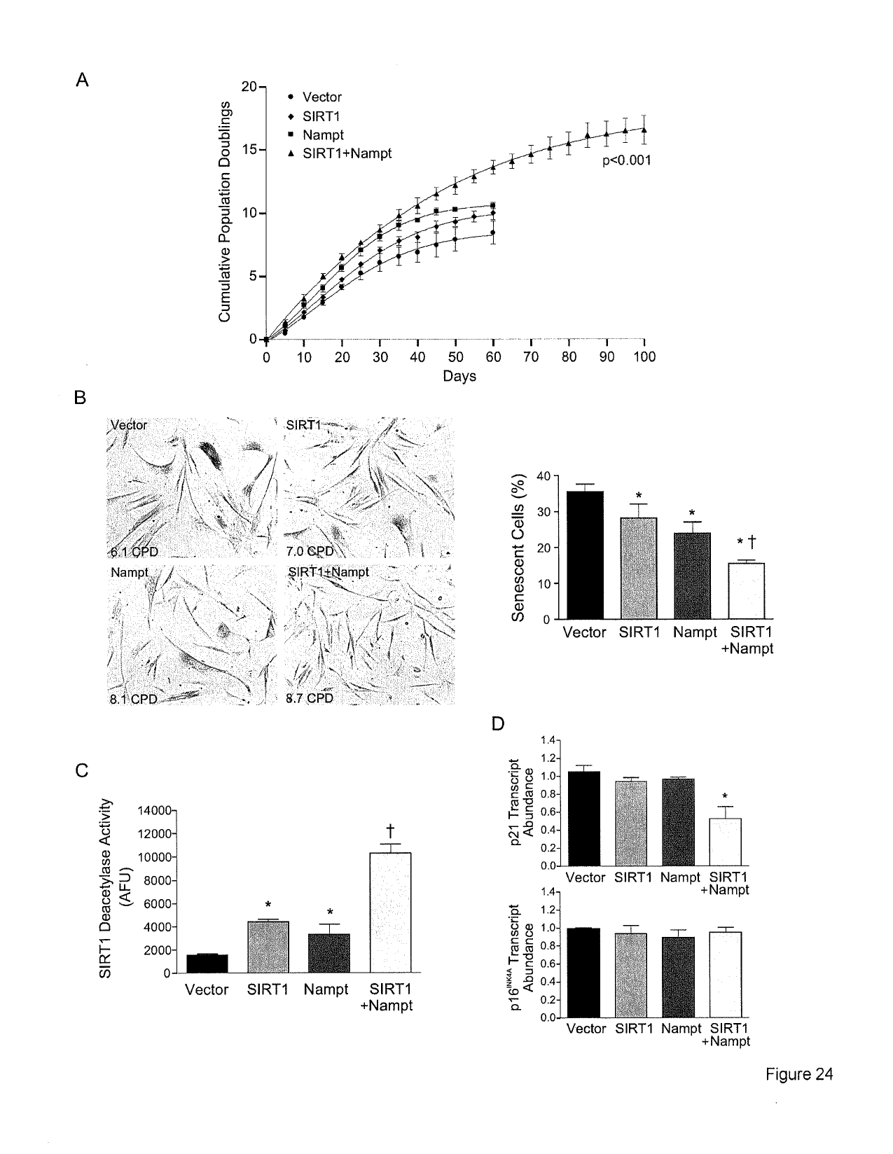

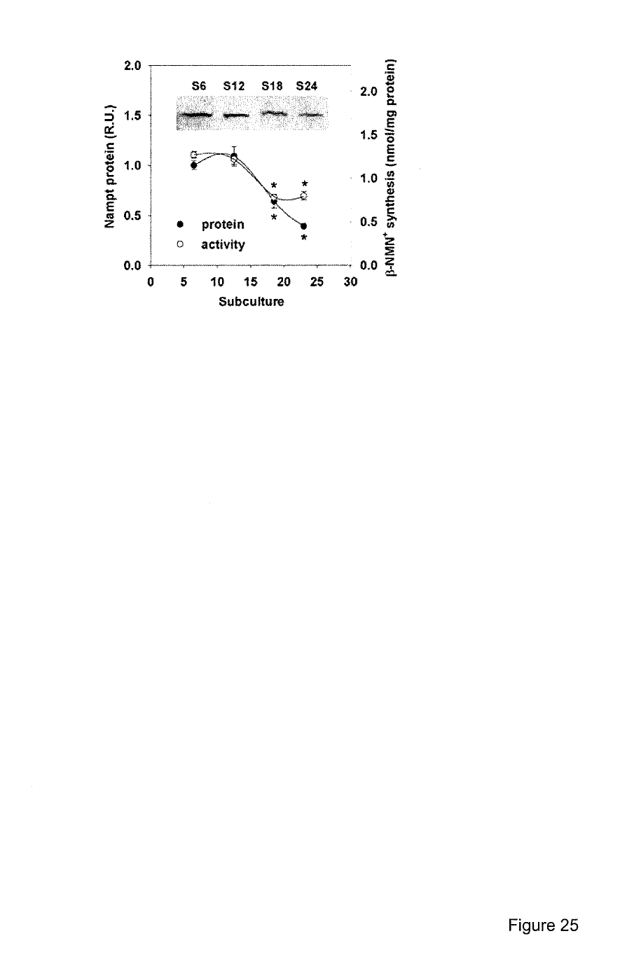

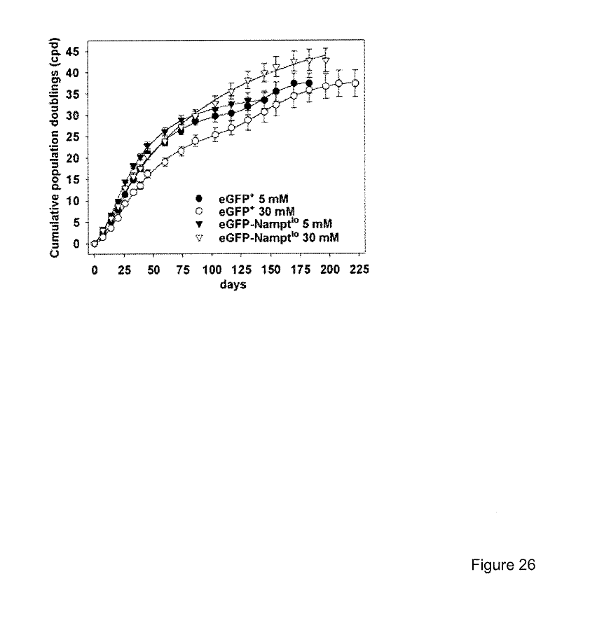

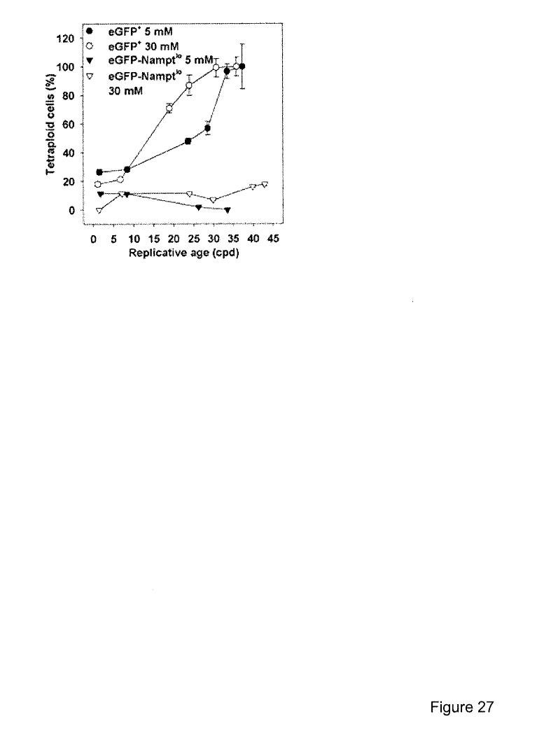

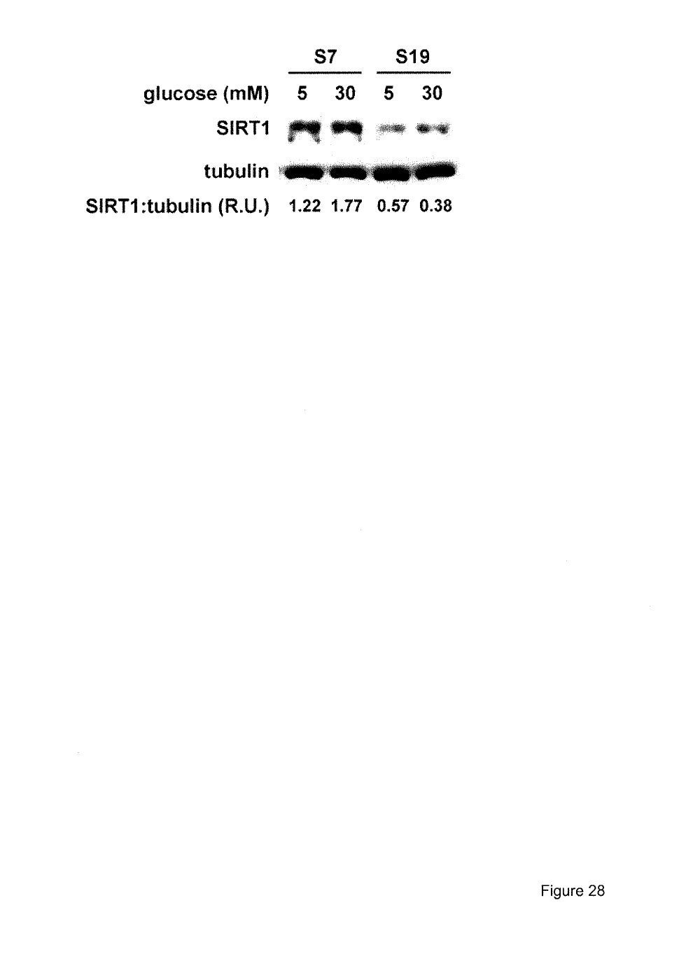

These screens suggested that PBEF was substantially upregulated as SMC's adopted a mature, contractile state. To verify this finding, HITB5 SMC's were analyzed for PBEF mRNA and protein expression by Northern and Western blot analysis. As shown in FIG. 1, six days after serum withdrawal, HITB5 SMC's converted from spread cells variably oriented on the dish to highly elongated cells that had crawled in a directed fashion into multilayered cell aggregates. Concurrently, the three major transcripts of PBEF (4.8, 2.9, and 2.2 kb) were substantially upregulated (FIG. 1b). Intracellular PBEF protein abundance also increased in maturing HITB5 and HITC6 SMC's, as did the expression of the contractile apparatus proteins, h-caldesmon and smoothelin A (FIG. 1c), confirming the relationship between PBEF expression and SMC maturation. PBEF was not detected in concentrated culture media at any stage of the maturation program (data not shown).

Overexpression of PBEF which increase Nampt activity, stimulates the maturation of smooth muscle cells. To determine if PBEF was functionally linked to SMC phenotype, we overexpressed PBEF in immature human SMC's. HITB5 SMC's were infected with retrovirus containing either pLNCX2-IRES-EGFP (HITB5-EGFP) or pLNCX2-PBEF-IRES-EGFP (HITB5-PBEF) and stable transductants were selected with neomycin. Under baseline, serum-supplemented conditions, SMC's overexpressing PBEF were longer and thinner than vector-infected SMC's. Elongation in response to PBEF was also observed with HITC6 SMC's and primary SMC's (data not shown). Furthermore, elongation of PBEF-overexpressing SMC's relative to control SMC's persisted as the cells further elongated in response to serum withdrawal (FIG. 2). To determine if the spatial organization of maturing SMC's was impacted by PBEF, cells were plated at higher densities (12,000 cells/cm²) and subjected to serum withdrawal. After 3 days, control SMC's had begun to aggregate although the extent of patterning was modest. In contrast, HITB5-PBEF-EGFP SMC's rapidly aggregated and by 3 days had already assembled into discrete, multi-layered ridges and nodules (FIG. 2).

To assess the effect of PBEF on SMC contractile protein expression, cells were studied by Western blot analysis. As shown in FIG. 3, HITB5-EGFP SMC's displayed the characteristic upregulation of PBEF following serum withdrawal, together with increased expression of h-caldesmon and smoothelin A. Interestingly, PBEF-overexpressing HITB5 SMC's displayed increased levels of h-caldesmon and smoothelin A under baseline, serum-supplemented conditions, assessed from cell lysates probed and exposed simultaneously with that of vector-infected SMC's. As well, smoothelin B, which was not detected in control SMC's, was expressed in PBEF-overexpressing SMC's. Withdrawal of serum from cultures of HITB5-PBEF SMC's lead to further upregulation of smoothelin A. Moreover, metavinculin expression was induced following serum withdrawal from PBEF-overexpressing SMC's but remained undetected in HITB5-EGFP SMC's. Thus, augmented expression of PBEF shifts the morphological and biochemical phenotype of SMC's closer to that of mature SMC's in the adult vessel wall. This similarity to contractile SMC's in vivo was especially strong when serum was removed from the culture environment.

Overexpression of PBEF enhances NAMPT activity and reduces the degree and extent of SMC apoptosis. To determine whether SMC growth was impacted by PBEF expression, SMC's in 5% serum were tracked over an 11-day period. As shown in FIG. 4(A), HITB5-PBEF SMC's accumulated faster than HITB5-EGFP SMC's, with a doubling time of 5.4 days versus 7.3 days, respectively (p<0.01). Similar results were seen with primary cultures of SMC's overexpressing PBEF (data not shown). To determine if this increase in cell accumulation was due to increased DNA synthesis, SMC's were incubated with ³H]thymidine and thymidine incorporation, relative to total DNA content, was assessed.

As shown in FIG. 4b, there was no detectable difference in thymidine incorporation between control and PBEF-overexpressing cells. To assess if SMC survival was affected by PBEF, apoptosis was assessed using TUNEL. This revealed that the proportion of apoptotic HITB5-PBEF SMC's was approximately half that of HITB5-EGFP SMC's (p<0.01, FIG. 4c). This improved survival was consistent with the appearance of PBEFoverexpressing SMC's, including their smoothly contoured cell surface and the paucity of culture debris.

siRNA-mediated PBEF knockdown impairs the survival and maturation of SMC's. We next determined if endogenous PBEF was required for SMC maturation. For this, HITC6 SMC's were infected with retrovirus containing cDNA encoding a hairpin-forming siRNA fragment. To ensure the siRNA responses reflected PBEF knockdown, 3 different targeting fragments were studied and both PBEF mRNA and protein were quantified. Two of the 3 siRNA constructs (siRNA147, siRNA1248) yielded a significant decrease in PBEF mRNA, quantified by real-time RT-PCR, compared to control SMC's infected with retrovirus containing cDNA encoding the corresponding non-silencing, RNA fragment (nsRNA147, nsRNA1248). SMC's expressing siRNA147 or siRNA1248 also showed significant suppression of PBEF protein. These SMC's had a short, truncated morphology (FIG. 5a) and they survived poorly, precluding serial passages. As well, the small fraction of PBEF-knockdown SMC's that remained adhered to the culture dish did not elongate following serum withdrawal (FIG. 5b). Heavy-caldesmon expression was also significantly lower in PBEF-knockdown SMC's than control SMC's (FIG. 5c). The limited cell viability precluded assessing h-caldesmon expression in response to serum withdrawal. Overall however, the poor cell survival, the perturbed morphology and inability to elongate, and the low expression of h-caldesmon in surviving SMC's indicate an inability of PBEF-knockdown SMC's to mature in culture. In contrast, SMC's expressing the siRNA construct (siRNA384) that did not manifest a reduction in PBEF mRNA or protein maintained an elongated morphology (FIG. 5a) and responded to serum withdrawal normally.

PBEF Increases Intracellular NAD⁺. In view of the controversy over human PBEF function and whether it acts intracellular or extracellularly, we generated a phylogenetic tree using protein sequences similar to human PBEF, derived from a Blast search of the NCBI sequence database. Multiple sequence alignment generated using ClustalW revealed that sequences from diverse species, bacterial and eukaryotic, were similar in length, contained strongly conserved regions with respect to helix propensity and hydrophobicity, and a conserved phosphoribosyltransferase domain. As shown in FIG. 6, the phylogenetic tree using selected sequences revealed very low point-assisted mutation scores, reflecting the short distances amongst all nodes and leaves of the tree. Thus, the PBEF protein has been well conserved throughout evolution which suggests a fundamental and invariant role. As this role has been shown in bacteria and rodent cells to involve NAD⁺ biosynthesis (10, 12), we next determined if the level of NAD⁺ in human cells was affected by PBEF. Analysis of cellular nucleotides by HPLC revealed that NAD⁺ content in HEK293 cells stably expressing the PBEF transgene was significantly higher than control cells (1.60±0.15 vs 0.85±0.08 μmol/10⁶ cells, p<0.01). Likewise, NAD⁺ content in HITC6-PBEF SMC's was greater than that in HITC6-Vector SMC's (1.90±0.02 vs 1.31±0.04 μmol/10⁶ cells, p<0.01) (FIG. 7).

NAD⁺-Dependent HDAC Activity is Increased in SMC's Overexpressing PBEF. Having established that PBEF increases intracellular NAD⁺ content in SMC's, we considered how this might impact SMC performance. NAD⁺-consuming reactions that depend on NAD⁺ regeneration include the deacetylation of certain histones and other proteins, postranslational modifications critical to gene silencing and cell survival (16, 17). To determine if PBEF influenced histone-deacetylase (HDAC) activity in SMC's, lysates from control and PBEF-overexpressing SMC's were incubated with [³H]-acetylated histone H4 peptide and HDAC activity quantified. As shown in FIG. 8, total HDAC activity was significantly greater in HITC6-PBEF SMC's than HITC6-Vector SMC's. To determine the relative amount of NAD⁺-dependent HDAC activity, deacetylase reactions were performed in the presence of 50 μM sirtinol, a noncompetitive inhibitor of NAD⁺-dependent (Class III) HDACs. Sirtinol significantly inhibited HDAC activity in PBEF-overexpressing SMC's, with a more modest inhibition of HDAC activity in control SMC's such that there was no longer a difference in HDAC activity between control and PBEF-overexpressing cells. We also examined the effect of 40 nM trichostatin A (TSA), an inhibitor of Class I and II HDACs, but not the NAD⁺-dependent HDACs. This substantially inhibited HDAC activity in both control and PBEF-overexpressing SMC's. However, the residual TSA-independent HDAC activity remained significantly greater in PBEF-overexpressing SMC's than in control SMC's. Taken together, the findings indicate that PBEF increases HDAC activity in human vascular SMC's and most, if not all, of this increase can be attributed to NAD⁺-dependent HDAC activity.

SMC's overexpressing PBEF, increases Nampt Activity Enhances Investment of Newly Formed Blood Vessels In Vivo. In order to determine if the survival and maturation profile observed in PBEFoverexpressing SMC's in vitro could translate into enhanced SMC performance in vivo, we studied SMC-based remodeling of newly formed blood vessels. During angiogenesis, SMC's wrap around the nascent vessel and assume the specialized phenotype. This investment process both stabilizes the microvessel and provides the machinery for vasomotor control (3, 18). To assess the integration of SMC's into the nascent vasculature, we developed a human-mouse chimeric model of angiogenesis. Growth factor-reduced matrigel mixed with FGF-2 and either PBEF-overexpressing (HITC6-PBEF-EGFP) or vector-transduced HITC6 SMC's (HITC6-EGFP) was injected subcutaneously into the abdominal regions of SCID mice. After eight days, mice were sacrificed and zinc-fixed, paraffin-embedded sections were studied histologically. By design, most of the interstitial cells in the implants were xenotransplanted human SMC's. Compared to control human SMC's, PBEF-overexpressing SMC's displayed greater immunoreactivity for the SMC maturation markers, h-caldesmon and calponin (FIG. 9(A)-(D)). Vascular integration of transplanted SMC's was determined by doubleimmunolabeling for mouse endothelial cells (anti-CD31) and human SMC's (anti-EGFP). Immunostaining for EGFP, as opposed to assessing EGFP fluorescence, proved to be a more sensitive detection system for SMC's in paraffin-embedded tissue. Paraffin processing, in turn, was critical to maintaining tissue architecture so that investment of endothelial-lined channels by SMC's could be unequivocally determined. As illustrated in FIG. 9e-i, a small proportion (approximately 2-5%) of human SMC's associated with endothelial cells. SMC's that invested microvessels assumed an elongated morphology with a more compact and elongated nucleus compared to SMC's in the interstitium. The proportion of microvessels that were invested by one or more human SMC's was significantly higher in matrigel implants containing PBEF-overexpressing SMC's (17.8±2.5%) than in implants loaded with vector-infected SMC's (10.7±2.2%, p<0.05) (FIG. 9j). PBEF-overexpressing SMC's could be found aligned with the long axis of microvessels, partially apposed to the endothelial cell as if actively extending to form the wall, or wrapped circumferentially around the microvessel (FIG. 9). Thus, SMC's with augmented capacity for NAD⁺ biosynthesis responded to the angiogenic environment in a specialized manner and integrated into the vasculature more efficiently than control SMC's.

With respect to FIG. 1, we show that PBEF is upregulated during maturation of HITB5 and HITC6 human vascular SMC's. FIG. 1(A) shows Hoffman-modulated contrast images of HITB5 smooth muscle cells in M199 supplemented with 10% FBS (left images) and 6 days after culturing SMC's in serum-free M100 (right image);

FIG. 1(B) is a Northern Blot showing up-regulation of the 3 major transcripts of PBEF in HITB5 SMC's following withdrawal of serum from cultures. FIG. 1(C) is a Western blot of cell lysates harvested from HITC6 SMC's before and after withdrawal of serum from cultures. Expression of PBEF protein increases as does expression of the SMC differentiation markers, h-caldesmon and smoothelin A.

With respect to FIG. 2, we show that overexpression of PBEF in human SMC's induces cellular elongation and aggregation of cells into multilayered ridges. The images of FIG. 2 show the cellular elongation and aggregation of human Smooth Muscle Cells into multilayered ridges induced by the overexpression of PBEF. FIG. 2(A), shows Hoffman-modulated contrast images of sub-confluent (top panel) or post-confluent (middle panel) HITB5 Smooth Muscle Cells transduced with a retrovirus containing cDNA encoding EGFP alone (left image) or PBEF and EGFP from a bicistronic cassette (right image). The bottom panel of FIG. 2(A) depicts fluorescence images of the post-confluent SMC cultures, showing expression of the transgenes as indicated by EGFP fluorescence. Bar, 50 μm. FIG. 2(B) shows the quantification of length-width ratios of control and PBEF-overexpressing Smooth Muscle Cells cultured in the presence of serum and 3 days after serum withdrawal. The dimensions of 50 randomly selected SMC's were determined using Northern Eclipse software (*p<0.01 vs HITB5-EGFP SMC's).

With respect to FIG. 3, we show that overexpression of PBEF in human SMC's stimulates expression of SMC differentiation proteins. FIG. 3 shows Western blots revealing expression of SMC differentiation markers in HITB5 SMC's infected with cDNA encoding EGFP alone (left image), and cDNA encoding both PBEF and EGFP (right image). Transductants were selected with G418 and lysates harvested before and on the designated days after serum withdrawal. Blots for control and PBEF-overexpressing SMC's were probed with a given antibody and exposed simultaneously.

With respect to FIG. 4, we demonstrate that PBEF reduces SMC apoptosis. FIG. 4(A) shows cell accumulation over 11 days for control and PBEF-overexpressing HITB5 SMC's, cultured in M199 with 5% FBS. Cell numbers from quadruplicate wells were quantified using a hemacytometer and the result shown is representative of 2 separate experiments (*p<0.01). FIG. 4(B) shows Thymidine incorporation into control and PBEF-overexpressing HITB5 SMC's, assessed by incubating cells in log-phase growth with 10 μCi/mL [³H]thymidine for 12 hours. Thymidine incorporation is expressed relative to cellular DNA content, determined by fluorescence spectrometry of Hoechst 33258-stained lysates. FIG. 4(C) shows fluorescence images of control and PBEF-overexpressing SMC's stained with Hoechst 33258 to identify nuclei (top panel) and for apoptotic nuclei by incubating with d-UTP fluorescein (bottom panel). SMC's were plated on glass slides, cultured in M199 with 10% FBS, and fixed with 4% paraformaldehyde. *p<0.01 vs control HITB5 SMC's.

With respect to FIG. 5, we demonstrate that knock-down of PBEF expression reduces the viability of Smooth Muscle Cells, and prevents serum withdrawal-induced maturation. The images of FIG. 5 show the effect on SMC viability of the knockdown of PBEF expression and maturation induced by serum withdrawal. FIG. 5(A) shows Hoffman-modulated images of control HITC6 SMC's and HITC6-siRNA SMC's. Western blots showing PBEF protein expression for each cell line are shown. HITC6 SMC's were transduced with the pSIREN-RetroQ vector containing a non-silencing oligodeoxynucleotide (HITC6 nsRNA) or an oligodeoxynucleotide encoding a hairpin siRNA fragment (HITC6siRNA). Fragments were 19 nucleotides in length beginning at nucleotides 147, 384, and 1248 from the start of the coding sequence. Transductants were selected with puromycin. FIG. 5(B) shows the length-width ratios of 50 randomly selected cells expressing either nsRNA 1248 or siRNA 1248. B. Length-width ratios were determined for cells in M199 with 10% FBS and 3 days after serum withdrawal. *p<0.05 vs control SMC's expressing the non-silencing RNA. FIG. 5(C) is a Western blot showing reduced expression of hcaldesmon in HITC6-siRNA 1248 SMC's.

FIG. 6 shows that human PBEF is a phylogenetically conserved nicotinamide phosphoribosyltransferase. FIG. 6 depicts a phylogenetic tree showing a tight evolutionary relationship between bacterial nicotinamide phosphoribosyltransferases and eukaryotic PBEF, including human PBEF. Sequences bearing similarity to the full length H. sapiens PBEF protein were used to generate a phylogenetic tree which establishes a tight evolutionary relationship between bacterial nicotinamide phosphoribosyltransferases and eukaryotic PBEF, including human PBEF. A representative selection of organisms were chosen for illustration and expect (E) values for the designated sequence alignments are shown. The point accepted mutation matrix (PAM) distances between the leaves and nodes of the phylogenetic tree are also shown. Sequences depicted are: H. sapiens (gi|1172027); M. musculus (gi|113278525); R. norvegicus (gi|29293813); X. laevis (gi|28278775); S. domuncula (gi|6689202); H. ducreyi (gi|33152518).

The depictions of FIG. 7 show the effects of increasing PBEF levels on the levels of NAD⁺. FIG. 7(A) shows the HPLC analyses of deproteinized nucleotide extracts obtained from HEK293 cells transfected with pQCXIP or pQCXIP-PBEF, and HITC6 SMC's transduced with pQCXIP or pQCXIP-PBEF. NAD⁺, eluted from the column after approximately 10 minutes, is indicated by the arrow on the chromatograms. Quantitative data from 3 separate experiments for each cell type are shown in FIG. 7(B). Representative PBEF expression for control and PBEF-overexpressing cells is shown in the Western blot insets. The left lane of each blot depicts the control SMC's. *p<0.01 vs control cells.

FIG. 8 shows that increasing the levels of PBEF, increases NAD⁺-dependent histone deacetylase activity in human Smooth Muscle Cells. HITC6 SMC's were stably transduced with pQXCIP-PURO or pQXCIP-PBEF-PURO and HDAC activity determined in cell lysates (25 μg of protein), using [³H]histone H4 peptide as the substrate. The reactions were quenched by acid hydrolysis and catalytically released [³H]-acetate was extracted in ethyl acetate. Deacetylation reactions were performed in the presence of vehicle, 50 μM sirtinol, or 40 nM trichostatin A. *p<0.01 vs vector-infected SMC's under the same assay conditions.

With respect to FIG. 9, Smooth Muscle Cells overexpressing PBEF maintain a mature phenotype in vivo and promote vessel chimerism and SMC investment. FIGS. 9(A) through 9(D) show sections of matrigel implants loaded with human SMC's. FIGS. 9(E) through 9(I) show sections stained with h-caldesmon and h-calponin. FIG. 9(J) illustrates the quantification of the proportion of microvessels invested by at least one EGFP-positive SMC.HITC6 SMC's were stably transduced with pLNCX2-EGFP or pLNCX2-PBEF-EGFP, mixed with matrigel and 250 ng/ml FGF-2 and transplanted beneath the skin of SCID mice. The matrigel implants were harvested 8 days later and paraffin-embedded sections studied by immunohistochemistry. FIGS. 9(A-D), show sections of matrigel implants that had been loaded with either control or PBEF-overexpressing human SMC's, immunostained for human hcaldesmon (A, B) or calponin (C, D). Implants are populated by newly formed microvessels and xenotransplanted human SMC's. Staining of h-caldesmon and hcalponin is more prominent in implants containing PBEF-overexpressing SMC's (arrows). In FIG. 9(E-I), sections of zinc-fixed matrigel implants double-immunolabeled for endothelial cells (anti-mouse CD31) and human SMC's (anti-GFP). Bound anti-CD31 antibody was identified using DAB chromogen (brown color) and bound anti-EGFP antibody was visualized using red alkaline phosphatase substrate (red color). A proportion of newly formed blood vessels are invested by exogenously added human SMC's and this is especially prominent for implants containing PBEF-overexpressing SMC's (arrows). FIGS. 9(G-I) show high-magnification images showing intimate apposition of EGFP-positive, PBEFoverexpressing SMC's with mouse endothelial cells. In FIG. 9(G), a human SMC is shown aligned parallel to an endothelial cell-lined vessel containing red blood cells and leukocytes. FIG. 9(H) shows the partial contact of an elongated human SMC with an endothelial cell, possibly reflecting active investment of the microvessel by the transplanted SMC. In FIG. 9(I), which corresponds to the box in F, a human SMC is circumferentially wrapped around the mouse microvessel. All sections were counterstained with Harris' hematoxylin. Bar, 50 μm. J. Quantification of the proportion of microvessels invested by at least one EGFP-positive SMC. (*p<0.05 vs HITC6-EGFP-loaded gels).

FIGS. 12 A-C are phase contrast photomicrographs, at 20× magnification, of vector-transduced or PBEF-overexpressing HITC6 SMC's in response to treatment with 5-phosphoribosyl-pyrophosphate (PRPP), demonstrating that the PRPP increases the health of smooth muscle cells compared to control cells. FIGS. 12(A-C) show cells infected with vector HITC6 with vehicle that have been cultured in M199 containing 1% FBS for 24 hours prior to addition of 250 uM PRPP. In FIG. 12(A), HITC6-infected SMC's with vehicle have been cultured in 1% FBS for 72 hours. In FIG. 12(B), the SMC's have been cultured with PBEF plus PRPP. In FIG. 12(C), the SMC's have been cultured with PBEF plus PRPP plus 1 mM Nam.

FIGS. 13 A-B are phase contrast photomicrographs at 20× magnification of vector-transduced HITC6 SMC's in response to treatment with 5-phosphoribosyl-pyrophosphate (PRPP), demonstrating that the PRPP increases the health of smooth muscle cells compared to control cells. The cells were cultured were cultured in M199 containing 1% FBS for 24 hours prior to addition of 500 uM PRPP. FIG. 13(A) shows the results of HITC6-Vector with vehicle at 24 hours. FIG. 13(B) shows the results of HITC6-Vector plus PRPP at 24 hours.

FIGS. 14 A-B show the protective effects of providing Smooth Muscle Cells with PRPP. After 24 hours in a stressing low serum environment, cells given PRPP alone are protected from cell death and are generally more robust. FIGS. 14 A-B are phase contrast photomicrographs at 10× magnification of vector-transduced HITC6 SMC's in response to treatment with 5-phosphoribosyl-pyrophosphate (PRPP). SMC's were cultured in M199 1% FBS for 24 hours prior to addition of 500 uM PRPP. FIG. 14(A) shows the results of HITC6-Vector with vehicle at 24 hours. FIG. 14(B) shows the results of HITC6-Vector plus PRPP at 24 hours.

Smooth Muscle Cell Culture lines were employed to assess the role of PBEF. Experiments were performed using the maturation-competent human vascular SMC lines, HITB5 and HITC6, generated from the human internal thoracic artery, as described previously (5, 6). SMCs were maintained in M199 (GibcoBRL, Burlington, ON) supplemented with the designated concentration of FBS (Hyclone). HEK293 cells were grown in DMEM with 10% FBS.

Overexpression of PBEF in Human Smooth Muscle Cells was evaluated by use of a viral vector. A retroviral gene delivery system was used to generate human SMCs stably overexpressing PBEF. Full-length cDNA encoding PBEF was amplified from HITB5 SMC mRNA by RT-PCR and subcloned into the pIRES-EGFP vector (Clontech). The PBEF-IRES-EGFP bicistronic fragment was then excised using Ahol and NotI and inserted into the retroviral expression vector pLNCX2 (Clontech). A second retroviral expression construct was generated by inserting PBEF cDNA into pQCXIP-IRES-PURO (Clontech). Retrovirus containing the cDNA of interest was obtained by calcium phosphate-mediated transfection of the Phoenix-amphotropic retrovirus packaging cell line (kindly provided by Dr. G Nolan, Stanford University Medical School, CA, distributed by ATCC, Manassas, Va.) as described previously (29). Virus-containing supernatant was added to proliferating SMCs and stable transductants were selected with 500 μg/ml G418 for 14 days, for pLNCX2-based constructs, and with 3 μg/ml puromycin for 48 hours, for pQCXIP-based constructs. Overexpression of PBEF was confirmed before each experiment by Western blot analysis.

Western Blot Analyses were employed to assay marker expression. Expression of PBEF and SMC differentiation markers was assessed by Western blot analysis with chemiluminescence detection, as described (29). Equal amounts of protein were resolved on 12% (for PBEF and α-tubulin), 9% (for smoothelin A and smoothelin B) and 6% (for caldesmon and vinculin-metavinculin) SDS-polyacrylamide gels and transferred PVDF membranes (Immobilon, Millipore). PBEF was detected using a polyclonal rabbit antibody against human PBEF (1857, 1:5000, kindly provided by Amgen). Monoclonal antibodies were used to detect heavy (h)-caldesmon (clone hHCD, 1:1000, Sigma), smoothelin isoforms A and B (clone MAB3242, 1:500, Chemicon), vinculin-metavinculin (clone VIN-11-5, 1:2000, Sigma), and α-tubulin (clone B-5-1-2; 1:16000, Sigma).

Cell Proliferation, DNA Synthesis, and Apoptosis were also evaluated. To assess SMC proliferation, cells were plated at a density of 3000 cells per cm² and cultured in M199 containing 5% FBS. Triplicate wells were harvested at the designated times and counted using a hemacytometer. To quantify DNA synthesis, cells in log-phase growth were incubated for 12 hours with [³H]thymidine (10 μCi/mL) and TCA-precipitable counts determined as described (5). Thymidine incorporation was expressed relative to DNA content, quantified by spectrofluorimetry of an aliquot of cell lysate incubated with 500 μg/ml Hoechst 33258. Apoptosis was assessed in SMCs seeded on glass coverslips by in situ end-labeling of DNA fragments using terminal deoxynucleotide transferase and fluorescein 12-dUTP (Promega) (5). Cells were fixed with 4% paraformaldehyde and counterstained with Hoechst 33258.

Knockdown of PBEF by RNA Interference was evaluated with a viral vector. PBEF knockdown was accomplished by infecting human SMC's with retrovirus containing sequences encoding hairpin siRNA fragments. Complementary oligodeoxynucleotides were synthesized, annealed, and inserted between the BamH1 and EcoR1 sites of the retroviral expression vector pSIREN-RetroQ. Three different targeting sequences were used, each consisting of 19 nucleotides starting at nucleotides 147, 384, and 1248 of the PBEF coding sequence (siRNA147 5′-GGAAGGTGAAATATGAGGA-3′; siRNA384 5′-ATGTTCTCTTCACGGTGGA-3′; siRNA1278 5′-AGGGCCGATTATCTTTACA-3′). Each sequence was separated by a 9-nucleotide noncomplementary spacer from the reverse complement of the same 19-nucleotide sequence. Blast search confirmed that only the PBEF gene was targeted. Control inserts contained the gene-specific 19-nucleotide sequence and hairpin loop sequence but not the antisense component. Infection of SMCs was performed as described above and cells were selected using 3 μg/ml puromycin for 48 hours.

Real-time RT-PCR was used to analyze nucleic acid evidence. RNA was harvested using RNeasy mini-column and reagents (Qiagen) and subjected to DNaseI treatment. Probe (5′-CAGTTGCTGATCCCA-3′) and flanking primers (5′-primer 5′-TGCAGCTATGTTGTAACCAATGG-3′; 3′-primer 5′-ACAAAAGGTCGAAAAAGGGCC-3′) for Taqman real-time RT-PCR for PBEF were designed using Primer Express software (Applied Biosystems). Reactions were performed using an ABI-Prism 7900 Sequence Detection System (Applied Biosystems). Optimum signal was obtained with concentrations of 200 nM, 300 nM and 50 nM for the probe, 5′ primer, and 3′ primer, respectively. Standard curves were generated using RNA derived from human aortic SMCs, enabling correlation of the determined threshold cycle to transcript abundance. GAPDH transcript abundance was used as an endogenous RNA control (Assays on Demand Hs9999905, Applied Biosystems) to which PBEF transcript abundance was normalized.

NAD⁺ analysis was determined by HPLC. Cellular nucleotides were extracted using perchloric acid, neutralized with KOH, and stored at −80° C. (30). The deproteinized cell lysate residues were then analyzed by HPLC using a mobile phase of 10 mM KH₂PO₄, 0.12% di-n-butylamine, pH, 3.0. Sample was injected onto a Prodigy C8 column (150×3.2 mm, 5 μm) (Phenomenex, Torrance, Calif., USA) by a Hewlett Packard 1090 chromatograph. The column temperature and flow rate were maintained at 40° C. and 0.5 ml/min, respectively. The effluent was monitored at 260 nm by a Hewlett Packard 1050 UV-VIS detector and NAD⁺ retention time, determined from an NAD⁺ standard, was 10 minutes.

Histone Deacetylase (HDAC) Assays were employed. Histone H4 peptide (Upstate) was labeled with [³H]-acetyl Coenzyme A (ICN) using PCAF histone acetylase. Cell lysates were harvested using Passive Lysis Buffer (Promega) and 25 μg of protein was incubated at 37° C. for 6 hours with 50,000 cpm of [³H]-acetyl histone and 1 mM PMSF in HDAC Assay Buffer (Upstate). Released [³H]-acetate was extracted with ethyl acetate (31) and counted on a Beckman LS3801 scintillation counter.

Human-Mouse Chimeric Angiogenesis in vivo was measured. HITC6 SMCs in M199 with 10% FBS and fibroblast growth factor-2 (FGF-2) were mixed with an equal volume of growth factor-reduced matrigel (BD Discovery Labware) yielding final concentrations of 5×10⁵ cells/ml and 250 ng/ml FGF-2. The cell-matrigel suspension (500 μl) was subcutaneously injected into the abdomen of mice with severe combined immunodeficiency syndrome (SCID). After 8 days, implants were harvested, fixed for 8 h in Tris-buffered zinc (32), and paraffin-embedded tissues were sectioned at 5 μm. After deparaffinization, endogenous peroxidases were quenched with 0.3% H₂O₂, nonspecific binding was blocked with 5% goat serum and sections were immunostained for h-caldesmon, calponin (clone hCP, Sigma), CD31 (BD Pharmingen, Bedford, Mass.), and GFP (BD Clontech, Palo Alto, Calif.). To simultaneously visualize mouse endothelial cells and human SMCs, sections were incubated overnight at 4° C. with biotinylated rat anti-mouse CD31 and bound antibody reacted with ABC reagent (Vector labs, Inc., Burlingame, Calif.) and diaminobenzidine (DAB, Vector). Tissue was then incubated with rabbit anti-GFP (1:200, BD Clontech, Palo Alto, Calif.) and bound primary antibody detected with alkaline phosphatase-conjugated goat anti-rabbit secondary antibody and visualized with red alkaline phosphatase substrate (Vector). Single and double immunolabeled sections were counterstained with Harris' hematoxylin.

Microscopy and Image Analysis were used to measure cell robustness and function. Cell images were collected using a Zeiss Axiovert S100 microscope (Carl Zeiss Microimaging Inc.) equipped with Hoffman Modulation Contrast Plan objectives and TCT condenser (Modulation Optics Inc.), cooled QICAM 12-bit Mono Fast 1394 camera (QImaging Inc.) and Northern Eclipse image analysis software (Empix Imaging Inc.). Histology images were acquired with an Olympus BX50 microscope with a BX-FLA illuminator, UPlanF1 objective lenses (Olympus Optical Co. Ltd.), cooled Retiga EXi Mono Fast 1394 camera (QImaging Inc.) and Northern Eclipse image analysis software. Linear image processing was done using Photoshop CS (Adobe Systems Inc.).

Smooth Muscle Cell Culture lines were employed to assess the role of PBEF. Experiments were performed using the maturation-competent human vascular SMC lines, HITB5 and HITC6, generated from the human internal thoracic artery, as described previously (5, 6). SMCs were maintained in M199 (GibcoBRL, Burlington, ON) supplemented with the designated concentration of FBS (Hyclone). HEK293 cells were grown in DMEM with 10% FBS.

Overexpression of PBEF in Human Smooth Muscle Cells was evaluated by use of a viral vector. A retroviral gene delivery system was used to generate human SMCs stably overexpressing PBEF. Full-length cDNA encoding PBEF was amplified from HITB5 SMC mRNA by RT-PCR and subcloned into the pIRES-EGFP vector (Clontech). The PBEF-IRES-EGFP bicistronic fragment was then excised using XhoI and NotI and inserted into the retroviral expression vector pLNCX2 (Clontech). A second retroviral expression construct was generated by inserting PBEF cDNA into pQCXIP-IRES-PURO (Clontech). Retrovirus containing the cDNA of interest was obtained by calcium phosphate-mediated transfection of the Phoenix-amphotropic retrovirus packaging cell line (kindly provided by Dr. G Nolan, Stanford University Medical School, CA, distributed by ATCC, Manassas, Va.) as described previously (29). Virus-containing supernatant was added to proliferating SMCs and stable transductants were selected with 500 μg/ml G418 for 14 days, for pLNCX2-based constructs, and with 3 μg/ml puromycin for 48 hours, for pQCXIP-based constructs. Overexpression of PBEF was confirmed before each experiment by Western blot analysis.

Western Blot Analyses were employed to assay marker expression. Expression of PBEF and SMC differentiation markers was assessed by Western blot analysis with chemiluminescence detection, as described (29). Equal amounts of protein were resolved on 12% (for PBEF and α-tubulin), 9% (for smoothelin A and smoothelin B) and 6% (for caldesmon and vinculin-metavinculin) SDS-polyacrylamide gels and transferred PVDF membranes (Immobilon, Millipore). PBEF was detected using a polyclonal rabbit antibody against human PBEF (1857, 1:5000, kindly provided by Amgen). Monoclonal antibodies were used to detect heavy (h)-caldesmon (clone hHCD, 1:1000, Sigma), smoothelin isoforms A and B (clone MAB3242, 1:500, Chemicon), vinculin-metavinculin (clone VIN-11-5, 1:2000, Sigma), and α-tubulin (clone B-5-1-2; 1:16000, Sigma).

Cell Proliferation, DNA Synthesis, and Apoptosis were also evaluated. To assess SMC proliferation, cells were plated at a density of 3000 cells per cm² and cultured in M199 containing 5% FBS. Triplicate wells were harvested at the designated times and counted using a hemacytometer. To quantify DNA synthesis, cells in log-phase growth were incubated for 12 hours with [³H]thymidine (10 μCi/mL) and TCA-precipitable counts determined as described (5). Thymidine incorporation was expressed relative to DNA content, quantified by spectrofluorimetry of an aliquot of cell lysate incubated with 500 μg/ml Hoechst 33258. Apoptosis was assessed in SMCs seeded on glass coverslips by in situ end-labeling of DNA fragments using terminal deoxynucleotide transferase and fluorescein 12-dUTP (Promega) (5). Cells were fixed with 4% paraformaldehyde and counterstained with Hoechst 33258.

Knockdown of PBEF by RNA Interference was evaluated with a viral vector. PBEF knockdown was accomplished by infecting human SMC's with retrovirus containing sequences encoding hairpin siRNA fragments. Complementary oligodeoxynucleotides were synthesized, annealed, and inserted between the BamH1 and EcoR1 sites of the retroviral expression vector pSIREN-RetroQ. Three different targeting sequences were used, each consisting of 19 nucleotides starting at nucleotides 147, 384, and 1248 of the PBEF coding sequence (siRNA147 5′-GGAAGGTGAAATATGAGGA-3′; siRNA384 5′-ATGTTCTCTTCACGGTGGA-3′; siRNA1278 5′-AGGGCCGATTATCTTTACA-3′). Each sequence was separated by a 9-nucleotide noncomplementary spacer from the reverse complement of the same 19-nucleotide sequence. Blast search confirmed that only the PBEF gene was targeted. Control inserts contained the gene-specific 19-nucleotide sequence and hairpin loop sequence but not the antisense component. Infection of SMCs was performed as described above and cells were selected using 3 μg/ml puromycin for 48 hours.

Real-time RT-PCR was used to analyze nucleic acid evidence. RNA was harvested using RNeasy mini-column and reagents (Qiagen) and subjected to DNaseI treatment. Probe (5′-CAGTTGCTGATCCCA-3′) and flanking primers (5′-primer 5′-TGCAGCTATGTTGTAACCAATGG-3′; 3′-primer 5′-ACAAAAGGTCGAAAAAGGGCC-3′) for Taqman real-time RT-PCR for PBEF were designed using Primer Express software (Applied Biosystems). Reactions were performed using an ABI-Prism 7900 Sequence Detection System (Applied Biosystems). Optimum signal was obtained with concentrations of 200 nM, 300 nM and 50 nM for the probe, 5′ primer, and 3′ primer, respectively. Standard curves were generated using RNA derived from human aortic SMCs, enabling correlation of the determined threshold cycle to transcript abundance. GAPDH transcript abundance was used as an endogenous RNA control (Assays on Demand Hs9999905, Applied Biosystems) to which PBEF transcript abundance was normalized.

NAD⁺ analysis was determined by HPLC. Cellular nucleotides were extracted using perchloric acid, neutralized with KOH, and stored at −80° C. (30). The deproteinized cell lysate residues were then analyzed by HPLC using a mobile phase of 10 mM KH₂PO₄, 0.12% di-n-butylamine, pH, 3.0. Sample was injected onto a Prodigy C8 column (150×3.2 mm, 5 μm) (Phenomenex, Torrance, Calif., USA) by a Hewlett Packard 1090 chromatograph. The column temperature and flow rate were maintained at 40° C. and 0.5 ml/min, respectively. The effluent was monitored at 260 nm by a Hewlett Packard 1050 UV-VIS detector and NAD⁺ retention time, determined from an NAD⁺ standard, was 10 minutes.

Histone Deacetylase (HDAC) Assays were employed. Histone H4 peptide (Upstate) was labeled with [³H]-acetyl Coenzyme A (ICN) using PCAF histone acetylase. Cell lysates were harvested using Passive Lysis Buffer (Promega) and 25 μg of protein was incubated at 37° C. for 6 hours with 50,000 cpm of [³H]-acetyl histone and 1 mM PMSF in HDAC Assay Buffer (Upstate). Released [³H]-acetate was extracted with ethyl acetate (31) and counted on a Beckman LS3801 scintillation counter.

Human-Mouse Chimeric Angiogenesis in vivo was measured. HITC6 SMCs in M199 with 10% FBS and fibroblast growth factor-2 (FGF-2) were mixed with an equal volume of growth factor-reduced matrigel (BD Discovery Labware) yielding final concentrations of 5×10⁵ cells/ml and 250 ng/ml FGF-2. The cell-matrigel suspension (500 μl) was subcutaneously injected into the abdomen of mice with severe combined immunodefiiency syndrome (SCID). After 8 days, implants were harvested, fixed for 8 h in Tris-buffered zinc (32), and paraffin-embedded tissues were sectioned at 5 μm. After deparaffinization, endogenous peroxidases were quenched with 0.3% H₂O₂, nonspecific binding was blocked with 5% goat serum and sections were immunostained for h-caldesmon, calponin (clone hCP, Sigma), CD31 (BD Pharmingen, Bedford, Mass.), and GFP (BD Clontech, Palo Alto, Calif.). To simultaneously visualize mouse endothelial cells and human SMCs, sections were incubated overnight at 4° C. with biotinylated rat anti-mouse CD31 and bound antibody reacted with ABC reagent (Vector labs, Inc., Burlingame, Calif.) and diaminobenzidine (DAB, Vector). Tissue was then incubated with rabbit anti-GFP (1:200, BD Clontech, Palo Alto, Calif.) and bound primary antibody detected with alkaline phosphatase-conjugated goat anti-rabbit secondary antibody and visualized with red alkaline phosphatase substrate (Vector). Single and double immunolabeled sections were counterstained with Harris' hematoxylin.

Microscopy and Image Analysis were used to measure cell robustness and function. Cell images were collected using a Zeiss Axiovert S100 microscope (Carl Zeiss Microimaging Inc.) equipped with Hoffman Modulation Contrast Plan objectives and TCT condenser (Modulation Optics Inc.), cooled QICAM 12-bit Mono Fast 1394 camera (QImaging Inc.) and Northern Eclipse image analysis software (Empix Imaging Inc.). Histology images were acquired with an Olympus BX50 microscope with a BX-FLA illuminator, UPlanF1 objective lenses (Olympus Optical Co. Ltd.), cooled Retiga EXi Mono Fast 1394 camera (QImaging Inc.) and Northern Eclipse image analysis software. Linear image processing was done using Photoshop CS (Adobe Systems Inc.).

### EXAMPLE 2

### Extension of Human Cell Lifespan by Nicotinamide Phosphoribosyltransferase

SMC's in developing arteries, and a proportion of SMC's in diseased adult arteries, exist in an immature, non-contractile state. To mature into a phenotype found in the normal adult artery wall, these cells must exit the cell cycle, elongate, and acquire the capacity to contract in response to vasoactive stimuli. We have shown that this switch in SMC phenotype depends on the actions of PBEF. PBEF was substantially upregulated as human SMC's converted to the contractile state and was essential for the survival of SMC's in an environment that no longer supported SMC proliferation. Furthermore, increased expression of PBEF stimulated cellular elongation, increased the expression of multiple SMC marker genes, and promoted vascular maturation in vivo. This augmentation in SMC maturation by PBEF was associated with increased intracellular levels of intracellular NAD⁺ and increased NAD⁺-dependent histone deacetylase activity. As histone deacetylation is a is a product of SIRT1 activity, these findings collectively implicate PBEF-mediated NAD⁺ flux as a regulatory system for SMC phenotype.

The SMC differentiation program is characterized by the ordered expression of genes that encode proteins of the SMC contractile apparatus (2). The HITB5 and HITC6 SMC's used in this study express a number of these proteins, even in the proliferative, noncontractile phenotype, including smooth muscle α-actin, h-caldesmon, and smoothelin A (5). Thus, the shift to a contractile state for these cells, induced by serum withdrawal, represents a late phase of the SMC developmental program. Primary human SMC cultures typically are not capable of this shift and also do not survive in the absence of serum or exogenous survival factors. In addition, expression of PBEF is lower in primary human SMC cultures than in the maturation-competent clones used in this study (data not shown). In this context, our finding that SMC viability was compromised by PBEF knockdown implies that PBEF is, as a minimum, permissive for SMC maturation. That is, PBEF may ensure the cell's survival in an environment that does not support cell growth. In addition, overexpression of PBEF yielded elongated SMC's with elevated levels of h-caldesmon, smoothelin A, and smoothelin B, even in the presence of serum, suggesting that PBEF also participates more directly in the maturation program. Following serum withdrawal, SMC maturation was even more robust, evidenced by expression of metavinculin and the striking multicellular patterning. Thus, PBEF may influence SMC differentiation by both facilitative and stimulatory mechanisms. These actions may also be relevant to other cell types because upregulation of PBEF expression has been observed during maturation of dendritic cells and B-lymphocytes (7, 19, 20).

The molecular pathways by which PBEF acts have been controversial. PBEF was initially identified during a screen for cytokines and proposed to be a cytokine based on sequences in the 3′ untranslated region and on effects of recombinant PBEF on the formation of pre-B-lymphocyte colonies. In these studies, recombinant PBEF by itself did not influence colony formation and the effect was observed only after addition of stem cell factor and IL-7 (7). Support for the notion of PBEF as a cytokine has come from studies wherein recombinant PBEF, generated by bacteria, stimulated the expression of inflammatory genes (8, 21) and inhibited neutrophil apoptosis (9). Whether these effects were mediated by a cytokine receptor is unclear. Moreover, the assignment of PBEF as a cytokine has been challenged because PBEF has no coding sequence homology to cytokines, no signal sequence for secretion, and does not appear to be secreted from cells by either a classical or alternative pathway (10, 11). We were unable to detect PBEF protein in the concentrated conditioned media from PBEF-overexpressing cell lines despite substantial upregulation of intracellular protein.

In order to better understand the mechanism of Nampt role in cell survival, one must consider the pathway within which it functions. This includes the balancing of NAD+ and its metabolites.

A number of vital enzymatic reactions, including deacetylation of histones, utilize and consume NAD⁺ (22). Degradation of NAD⁺ in these non-redox reactions liberates nicotinamide, from which NAD⁺ can be regenerated via a two-step salvage pathway. Nicotinamide phosphoribosyltransferase/PBEF catalyzes the conversion of nicotinamide to nicotinamide mononucleotide (NMN). NMN is then converted to NAD⁺ by nicotinamide mononucleotide adenylyl-transferase-1 (Nmnat1) (14, 15). Interestingly, Nmnat1 has recently been found to increase NAD⁺ synthesis and prevent axonal degeneration in explanted mouse neurons (23). Furthermore, NAD salvage pathway genes in yeast and mammals have been found to activate the NAD⁺-dependent HDAC, Sir2 (or the mammalian ortholog, SIRT1). Sir2 (silent information regulator 2) is a NAD⁺-dependent longevity enzyme that deacetylates H3 and H4 histones, certain transcription factors, and p53 (22, 24-26).

The current findings with Nampt demonstrate that NAD⁺ regeneration impacts mammalian cell survival and specifically link Nampt with NAD⁺-dependent protein deacetylation, which is the mode of action of SIRTs. Moreover, the data establish that the functional consequences of NAD⁺ salvage; SIRT1 mediated modification of P53 activity, and BPEF activity with respect to cell survival.

The inventors have established that a programmed diminution in the capacity to regenerate NAD⁺ from nicotinamide is a critical precursor of human SMC senescence. Moreover, by enhancing Nampt activity, cellular lifespan can be lengthened, a phenomenon that was observed in human primary SMCs, human clonal SMCs, and fibroblasts derived from a patient with HGPS. The inventors have further established that this anti-aging phenomenon is mediated by enhanced SIRT1 deacetylase activity that, in turn, holds p53 levels below that which induce senescence. These findings implicate a Nampt-SIRT1 axis as a fundamental determinant of human cell aging.

The role of Nampt as a driver of cellular stress resistance and longevity is noteworthy in the context of its expression profile. Nampt is upregulated by stressful stimuli including infection (Jia, 2004) and pro-inflammatory cytokines (Kendal, 2006). In human SMCs, Nampt expression increased substantially in response to the stress of complete serum removal (van der Veer, 2005). The ability of Nampt to enhance resistance to stress and extend cellular longevity in a SIRT1-dependent manner suggests that Nampt may orchestrate an analogous longevity paradigm in human cells. The current data also suggest, however, that such a protective/longevity response to mild stress would itself be dependent on cellular age. That is, the endogenous decline in Nampt activity that was observed in pre-senescent cells might underlie a switch in the response to low level stress from lifespan extension to accelerated aging. Nampt expression and activity decline as human SMCs undergo replicative aging. To track Nampt expression as SMCs undergo replicative aging, two human SMC preparations with different in vitro lifespans were studied: 1) primary cultures of SMCs, initiated by outgrowth from the internal thoracic artery of adults with coronary atherosclerosis; and 2) a clonal line of human SMCs (HITC6) that also originated from the internal thoracic artery but displayed enhanced longevity in culture (7,14). SMCs were serially subcultured until they reached senescence, indicated by cessation of proliferation, an enlarged flattened morphology, and cytoplasmic β-galactosidase activity at pH 6.0 (FIGS. 17A and 17B). In both SMC preparations, Nampt protein expression declined as SMCs approached senescence (FIGS. 17C and 17D). In contrast, Nampt enzyme activity, assessed by quantifying the conversion of nicotinamide to nicotinamide mononucleotide (6), fell significantly in pre-senescent SMCs, much more strikingly than Nampt expression, reaching 14±3% (mean±SD, n=3) of basal activity (FIG. 17C). These findings identify the regeneration of NAD⁺ from nicotinamide as a metabolic pathway that becomes exhausted, by virtue of a pronounce decline in Nampt activity, as SMCs approach senescence.

Nampt regulates and extends human cellular lifespan. To determine if human SMC lifespan could be extended by overriding this innate decline in Nampt activity, the inventors augmented Nampt gene dosage by introducing human Nampt cDNA into SMCs using retrovirus. This yielded a 7.1±3.1-fold increase in Nampt activity in stably transduced SMCs. Primary adult SMCs overexpressing Nampt surpassed the maximal lifespan of vector-infected SMCs by an additional 2.1±0.3 population doublings which, given their otherwise short in vitro lifespan, constituted a 34±4% prolongation of lifespan (p=0.01, FIG. 18A). Equally notable, this extension proceeded in cells that were already well advanced along their path to replicative senescence. In the longer-lived HITC6 SMCs, lifespan extension by Nampt was even more striking, with an additional 6.3±0.3 population doublings, or a 71±7% extension of lifespan (p=0.003). Cell lifespan extension by Nampt was not limited to SMCs and was also seen with human fibroblasts derived from a subject with HGPS, a condition associated with markedly premature atherosclerosis (15) (FIG. 18A).

To determine the role of endogenous Nampt in SMC lifespan and senescence, SMCs were incubated with the specific Nampt antagonist FK866 (Hasmann, 2003). FK866 is a long, almost linear molecule that binds Nampt from within a narrow tunnel at the Nampt dimer interface. This unusual structural relationship accounts for the specificity of FK866 and this tunnel is not present even in the closely related dimeric enzyme, nicotinic acid phosphoribosyltransferase (Khan, 2006). The inventors thus quantified the proportion of senescent SMCs in successive subcultures incubated with 10 nM FK866, a concentration determined to reduce PBEF activity in SMCs to 22±2% of baseline. Kaplan-Meier survival analysis revealed a substantially shortened senescence-free survival of PBEF-inhibited SMCs (p<0.0001) (FIG. 18B). In contrast, there was a markedly extended senescence-free survival in PBEF-overexpressing SMCs vs. vector-infected cells (p<0.0001) (FIG. 18C). Therefore, a direct relationship exists between PBEF activity and the number of replication cycles a SMC can undergo before becoming senescent. Together with the age-related decline in PBEF activity, these data firmly establish PBEF as a longevity enzyme for human SMCs.

Nampt postpones senescence by activating SIRT1. To explore the mechanism by which Nampt regulates SMC lifespan, the inventors considered that Nampt both stimulates NAD⁺ production and consumes nicotinamide, positioning this enzyme as a potential regulator of the NAD⁺-dependent deacetylase, SIRT1. SIRT1 is the mammalian sirtuin with the highest sequence similarity to Sir2. It is a predominantly nuclear enzyme that is essential for mammalian development (McBurney, 2003; Cheng, 2003). SIRT1 consumes NAD⁺, is inhibited by nicotinamide, and mediates lifespan extension of caloric restricted animals (Cohen, 2004). It was previously unknown, however, whether augmenting SIRT1 expression could extend the lifespan of vascular SMCs as the role of SIRT1 in regulating the replicative lifespan of mammalian cells in general was uncertain. For example, Langley et al. (2002) found that SIRT1 overexpression in mouse embryo fibroblasts inhibited promyelocytic leukemia protein-induced senescence. In contrast, Michishita et al. (2005) found that replicative lifespan of human fibroblasts was unaffected by SIRT1 overexpression. Further confounding the question was the observation that mouse embryonic fibroblasts deficient in SIRT1 had extended, rather than shortened, replicative lifespans (Chua, 2005).

To test whether Nampt could stimulate SIRT1 activity in human SMCs, TSA-independent deacetylation of a fluorogenic SIRT1 substrate (Biomol) was quantified. As shown in FIG. 19A, SMCs overexpressing Nampt had an 86±4% (p=0.03, n=4) increase in SIRT1 activity. The inventors were surprised to find that PBEF overexpression also modestly increased abundance of SIRT1 protein (1.3±0.3-fold, p=0.02), although not enough to fully account for the increased SIRT1-mediated deacetylase activity (FIG. 19B). To determine if the lifespan extending actions of Nampt were mediated by the increased SIRT1 expression and activity, human SMCs were double transduced to express Nampt and a dominant-negative form of SIRT1 (H363Y) (Vaziri, 2001). This revealed that the extended lifespan conferred by Nampt was abrogated when the SIRT1 H363Y allele was co-expressed (5.4±0.5 vs. 0.3±0.3 of additional population doublings, p=0.003). Furthermore, the reduction in senescent SMCs in late-passage (37^{th}) PBEF-overexpressing SMCs (19±2%, p=0.003) was no longer observed when dominant-negative SIRT1 was co-expressed (p=0.3), with a relative increase in senescence that was greater than for vector-infected SMCs expressing SIRT1 H363Y (55±12 vs. 15±10%, p=0.01, FIG. 19C).

By tracking both expression and activity of SIRT1, the inventors found that SMC senescence is preceded by a marked decline in SIRT1 function that cannot be fully accounted for by a decline in SIRT1 expression. Consistent with this, overexpressing SIRT1 by itself only modestly extended SMC longevity. The inventors further established that this functional inhibition of SIRT1 in aging SMCs is because of a deteriorating NAD⁺-based metabolic milieu for sirtuin activity, and that profound SMC lifespan extension by SIRT1 can be achieved with strategies that optimise this milieu.

Nampt enhances p53 degradation and exogenous p53 abrogates Nampt-mediated inhibition of senescence. p53 is stabilized by acetylation of lysine 382 (K382) and this p53 site is a known target of SIRT1-mediated deacetylation (21). p53 promotes aging (22) and, in keeping with this, the inventors found that p53 abundance increased as human SMCs approached replicative senescence (FIG. 19D). However, this age-related increase in p53 was blunted in parallel cultures of SMCs overexpressing Nampt. Furthermore, the fraction of p53 that was acetylated on K382 was substantially lower in Nampt-overexpressing SMCs than in control cells (FIG. 19E). This p53 modification was associated with a significantly increased rate of p53 degradation in PBEF-overexpressing SMCs, as assessed in SMCs incubated with cycloheximide (FIG. 19F). To determine if altered p53 levels underlay the changes in senescence induced by PBEF, p53 was introduced to SMCs using recombinant adenovirus (23). As shown in FIG. 19G, add-back of p53 to Nampt-overexpressing SMCs abrogated the reduction in senescent SMCs afforded by augmented Nampt activity. Collectively, these findings indicate that Nampt postpones cellular senescence by ensuring ongoing and efficient degradation of p53.

Nampt protects against oxidative cell damage. Using timelapse microscopy, the inventors established that the additional replicative life conferred by Nampt was associated with a healthy, stress-resistant phenotype. Whereas vector-infected SMCs responded to 150 μM H₂O₂ by global retraction of plasma membrane, Nampt-overexpressing SMCs, which were matched for cumulative population doubling, largely maintained their morphology and ability to migrate (FIG. 20A). Concurrently, H₂O₂ induced a rapid increase in cytoplasmic p53 in control SMCs whereas the response was blunted in Nampt-overexpressing SMCs (FIG. 20B).

Cell Culture. Experiments were performed using primary human vascular SMCs derived by outgrowth from fragments of internal thoracic artery and the HITC6 SMC clonal line, also originally generated from the human internal thoracic artery (Li, 2001). Derman fibroblasts from an individual with Hutchinson-Gilford Progeria Syndrome (HGPS) were obtained from Coreill Cell Repository.

To quantify replication, cells were plated at 4500 cells/cm² and growth medium with 10% FBS was changed every two days until cells reached 90-95% confluence. Harvested cells were counted from triplicate plates and the number of population doublings was calculated based on: [log₁₀(number of cells harvested)−log₁₀(number of cells seeded)]/log₁₀ (Fukuhara, 2005). Population growth curves were compared using both nonlinear regression and two-way analysis of variance.

Recombinant Retrovirus and Adenovirus Infection. A retroviral gene delivery system was used to generate human cells stably overexpressing PBEF, using methods described previously (Rocnik, 2002). Retrovirus containing pQCXIP-PBEF-IRES-PURO or pQCXIP-IRES-PURO (Clontech Laboratories, Mountain View, Calif.) was generated by calcium phosphate-mediated transfection of the Phoenix-amphotropic retrovirus packaging cell line (ATCC, Manassas, Va.). Stable transductants were selected with puromycin (3 μg/ml). Dominant-negative SIRT1 (H363Y) was similarly transduced using the pBABE retroviral expression vector.

Recombinant adenoviral vectors carrying human p53 or EGFP expression cassettes were constructed, purified, and titred as previously described (Cregan, 2000). Experiments were performed at a MOI of 100 pfu/cell.

Western Blot and Immunoprecipitation Analysis. Protein expression was assessed by Western blot analysis with chemiluminescence detection, as previously described (Rocnik, 2002). PBEF was detected using a rabbit polyclonal anti-human PBEF/Pre-B-cell colony enhancing factor antibody (Bethyl Laboratories, Montgomery, Tex.). SIRT1 was detected using a rabbit polyclonal anti-SIRT1 antibody (Abcam, Cambridge, Mass.) and p53 and α-tubulin were detected with monoclonal antibodies (CO-1, Santa Cruz, Calif. and Clone B-5-1-1, Sigma, Oakville, ON, respectively). Cytoplasmic p53 was similarly assessed following cell fractionation (NucBuster, Novagen Minneapolis, Minn.). Identification of p53 that underwent NAD⁺-dependent removal of an acetyl group on residue K382 was determined by pretreating SMCs for 2 h with 5 μM trichostatin A (TSA), immunoprecipitating p53 from the cell lysate with a goat polyclonal antibody (R&D Systems, Minneapolis, Minn.), and immunoblotting using a rabbit polyclonal anti-acetylated p53 (K382) antibody (American Proteomics, Carlsbad, Calif.) and HRP-labelled donkey anti-rabbit IgG antibody (Amersham, Piscataway, N.J.).

Nicotinamide Phosphoribosyltransferase Activity. Whole cell lysates were reacted with 5 μM [carbonyl-¹⁴C]-nicotinamide (Sigma) and 0.5 mM phosphoribosylpyrophosphate in 10 mM NaH₂PO₄/Na₂HPO₄ buffer, pH 8.8 (Rongvaux, 2002). Labelled acetone-precipitable nicotinamide mononucleotide was quantified by scintillation counting.

Senescence-associated β-Galatosidase Activity Assay. SMCs on glass coverslips at approximately 70% confluence were fixed in 2% formaldehyde/0.2% glutaraldehyde in PBS for 3 minutes and incubated with X-gal-containing reaction mixture, as previously described (Dimri, 1995). Washed cells were stained with Hoechst 33258 (2.5 μg/ml) and the proportion of senescence-associated β-galactosidase (SA β-Gal)-positive cells quantified from 10 microscope fields of view (Olympus BX51, ×20 objective, approximately 1200 cells). Senescence-free survival was determined using Kaplan-Meier analysis of survival vs. replicative age.

In vivo assessment of SIRT1 deacetylase activity. Cell-based assessment of SIRT1-like enzymatic activity was performed using the Fluor de lys-SIRT1 substrate (Biomol, Plymouth Meeting, Pa.), as previously described (de Boer, 2006). SMCs in phenol-red free M199 with 5% FBS were incubated for 2 h with 5 μM TSA followed by addition of the fluorogenic substrate for 4 h. Signal was quantified by spectrofluorimetry (Wallac, Wellesley, Mass.) and normalized to total protein content.

Time-lapse analysis of SMC response to oxidative stress. The morphologic response to oxidative stress was dynamically assessed by digital time-lapse microscopy, using methods previously described (Fera, 2004). Hoffman-modulated contrast images (Axiovert S100; Carl Zeiss, Inc. Thornwood, N.Y.) were digitally acquired every 5 minutes over 3 h, beginning immediately after the addition of 150 μM H₂O₂ to SMCs in M199 containing 1% FBS, with or without 5 μM TSA.

With respect to FIG. 17A, the inventors show Hoffman-modulated contrast images of human HITC6 SMCs in a healthy and proliferating state (left) and after eight further subcultures at which time cell replication ceased and the cells were flat and spread (right).

With respect to FIG. 17B, the inventors show HITC6 SMCs incubated with X-gal solution at pH 6.0 for senescence-associated β-galactosidase (SA β-Gal) activity and photographed in a proliferating state (left) and after having become senescent eight subcultures later (right). The transition to senescence was associated with a decline in PBEF expression, indicated by Western blot analysis of lysates harvested from primary human SMCs (FIG. 17C) and clonal HITC6 SMCs (FIG. 17D), studied at designated subcultures (s) following harvesting from fragments of adult internal thoracic artery. PBEF activity in SMCs depicted in panel C, assessed by incubating lysates with [¹⁴C]nicotinamide and phosphoribosylpyrophosphate, is shown.

With respect to FIG. 18A, the inventors show cumulative population doubling curves of primary SMCs (left), HITC6 SMCs (middle), and HGPS fibroblasts (right) infected with retrovirus containing control cDNA (pQCXIP, Vector) or cDNA encoding PBEF. Stable transductants were selected with puromycin and each graph depicts the averaged results from three longevity assessments. Curves were fit and compared based on nonlinear regression analysis. Kaplan-Meier analysis of senescence-free survival for HITC6 SMCs incubated with or without the PBEF antagonist FK866 (10 nM) (FIG. 18B) and HITC6 SMCs stably transduced with either empty vector or with cDNA encoding PBEF (FIG. 18C). The number of SA β-gal-positive SMCs and the total number of SMCs (based on Hoechst 33258-staining) were determined for serial subcultures in ten randomly chosen fields (p<0.0001 for both survival plot pairs, log-rank test). Corresponding micrographs of SMCs stained for SA β-Gal activity, at the indicated cumulated population doublings, (cpd) are shown on the right.

With respect to FIG. 19A, the inventors show SIRT1 activity, measured by quantifying NAD⁺-dependent deacetylation of an acetylated p53 peptide substrate (Fluor de Lys-SIRT1) in the presence of TSA (p-0.03, two-tailed t-test). With respect to FIG. 19B, we show Western blot showing modestly increased expression of SIRT1 in PBEF-overexpressing SMCs. FIG. 19C shows the proportion of senescent SMCs in late-passage (37^{th} subculture) HITC6 SMCs double transduced with amphotropic retrovirus containing cDNA for PBEF of pQCXIP empty vector and SIRT1H363Y mutant or pBABE empty vector (*p<0.05 vs. PBEF cDNA⁻/SIRT1H363Y cDNA⁻, **p<0.001 vs. PBEF cDNA⁻/SIRT1H363Y cDNA⁻, †p<0.001 vs. PBEF cDNA⁺/SIRT1H363Y cDNA⁺, aNOVA with Bonferoni post-hoc test). With respect to FIG. 19D, the inventors show a Western blot showing upregulation of p53 as SMCs approach the end of their replicative lifespan but little increase in PBEF-overexpressing SMCs at the equivalent subcultures. FIG. 19E shows Western blots of total p53 and p53 acetylated on lysine 382. A 25% overload of PBEF-overexpressing SMC lysates was used, yielding similar total p53 content, to facilitate comparison between deacetylated p53 signals. With respect to FIG. 19F, the inventors show p53 degradation profile in HITC6-Vector and HITC6-PBEF SMCs, assessed by treating cultures with cycloheximide (10 μg/ml) and TSA (5 μM). Degradation kinetics were fit with mono-exponential decay curves, averaged from three experiments, and statistical comparison between slopes (t-test, two-tailed) was made after natural logarithm transformation (inset) (p=0.03). With respect to FIG. 19G, the inventors show that the proportion of senescent SMCs in HITC6 SMCs double transduced with amphotropic retrovirus containing cDNA for PBEF or pQCXIIP empty vector and adenovirus containing p53 or EGFP (*p<0.05 vs. PBEF cDNA⁻/p53 cDNA⁻, **p<0.001 vs. PBEF cDNA⁻/p53 cDNA−, †p<0.001 vs. PBEF cDNA⁺/p53 cDNA⁻, ANOVA with Bonferoni post-hoc test).

With respect to FIG. 20A, the inventors show Hoffman-modulated contrast images of control (HITC6-Vector) and PBEF-overexpressing (HITC6-PBEF) SMCs immediately after and 2.5 h after addition of 150 μM H₂O₂ to media containing 1% FBS and 5 μM TSA. Side-by-side images dpict identical fields of view, demonstrating the pronounced degradation of morpohology of individual cells in control but not PBEF-overexpressing SMCs. With respect to FIG. 20B, accumulation of cytoplasmic p53 is seen within 1 h in control SMCs but minimally in PBEF-overexpressing SMCs.

### EXAMPLE 3

### Extension of Human Vascular SMC Lifespan by Multilevel Modulation of SIRT1

Enhancing expression of SIRT1 by itself may not be an effective strategy to extend SMC lifespan, despite a pronounced senescence-related decline in the activity of SIRT1. However, the inventors have found that the replicative lifespan of SMCs can be extended markedly if SIRT1 enhancement is paired with that of Nampt. The value of coordinating the expression of SIRT1 and Nampt is because the activity of SIRT1 in SMCs is highly dependent on Nampt-mediated NAD⁺ biosynthesis, and because Nampt activity is vulnerable and declines as a consequence of aging. Therefore, SIRT1 is a determinant of SMC lifespan but its activity, and SMC longevity itself, is ultimately defined by the level of Nampt activity.

The present inventors show that SIRT1 protein levels decline as human SMCs undergo serial cell passage and permanently withdraw from the cell cycle. In addition, the inventors have also established that SIRT1 activity declines significantly more than SIRT1 protein expression. This means that a feature of replicative senescence is the emergence of a functional inhibitor of SIRT1 activity and that SIRT1 activity, more so than expression, should be considered as a biomarker for vascular senescence.

The inventors have established that the senescence-related functional inhibition of SIRT1 was due to an innate decline in the activity of Nampt. Nampt is the rate-limiting enzyme for the conversion of nicotinamide to NAD⁺, and this conversion is important for two reasons. First, NAD⁺ is required for SIRT1-mediated deacetylation of its target protein because it supplies the necessary ADP ribose moiety to accept the acetyl group from the targeted protein. Consumption of NAD⁺ in this reaction means that its regeneration is critical. Second, hydrolysis of NAD⁺ releases nicotinamide, which is a known inhibitor of SIRT1 activity. Nicotinamide can be expected to accumulate if Nampt activity falls, with a resulting inhibition of SIRT1 activity.

Treatment of Vascular Disease. Cell senescence is strongly implicated in age-related pathologies as well as the recognized decline in tissue regenerative potential with age. Vascular SMC senescence, a hallmark of atherosclerotic lesions, can be particularly dangerous because the resulting pro-inflammatory phenotype and non-reparative state can incite lesion disruption and acute vascular occlusion. The identification of the molecular pathways that enable SMCs to resist premature senescence would have important implications for vascular disease. The current findings identify PBEF as underlying an aging suppression pathway in SMCs, with potential relevance to controlling atherosclerosis and possibly other diseases of aging.

The present methods and formulations are adaptable in a myriad of ways to provide cyto-protection and healing to cells and tissues. As those of skill in the pharmaceutical or cosmetic arts will comprehend, numerous combinations and formulations of PBEF and its activators, SIRTUINS and their activators, PRPP, and nicotinamide are within the scope and spirit of the invention, as are numerous variations of the present methods.

Introducing SIRT1 into Human SMCs Modestly Extends Replicative Lifespan. To determine if the replicative lifespan of human vascular SMCs was impacted by enhanced expression of SIRT1, the inventors infected proliferating human SMCs with retrovirus containing SIRT1 cDNA. Population doublings of stable transductants were then tracked until proliferation ceased, a transition concurrent with accumulation of flattened and enlarged SMCs indicative of senescence. In primary cultures of adult SMCs, which are generally short-lived, there was a modest (1.30±0.93-fold, p<0.01) increase in cumulative population doublings in SIRT1-overexpressing cells (SIRT1-SMCs) compared to vector infected SMCs (FIG. 21a). In a clonal line of non-immortal but longer-lived human SMCs, 13-15, the number of population doublings in SIRT1-SMCs increased by 1.17±0.03-fold (p<0.05) (FIG. 21b). Thus, introducing the SIRT1 gene into human SMCs extends replicative longevity, but modestly.

SIRT1 Activity Declines Precipitously in Aging SMCs. In view of the small effect of SIRT1 overexpression on replicative lifespan, the inventors sought to clarify the relationship between SMC aging, SIRT1 expression, and SIRT1 activity. Clonal, non-immortalized SMCs were serially subcultured until they became senescent, evident by the features described above. As shown in FIG. 22, this progression to senescence was associated with a decline in the expression of SIRT1. There was also a senescence-related decline in the activity of SIRT1, measured by the TSA-independent ability to deacetylate an exogenous substrate (BioMol). However, the decline in SIRT1 activity was much more striking than the decline in SIRT1 protein abundance. About three subcultures before global population senescence, SIRT1 activity fell precipitously, by 85±6% (p<0.01), whereas the corresponding reduction in SIRT1 protein abundance (44±13%) was significantly less (p<0.05) (FIG. 22). Consistent with the fall in SIRT1 activity were significant increases in the expression of p53 and p21 (p<0.05), two prosenescence proteins whose expression is repressed by SIRT1-mediated deacetylation.

SIRT1 Activity Declines in Aging SMCs Because of Exhaustion of the NAD⁺ Salvage Reaction. To ascertain why the activity of SIRT1 fell more strikingly than SIRT1 expression, the inventors considered the fact that SIRT1 is an NAD+-consuming deacetylase and the deacetylation reaction is inhibited by the NAD+hydrolysis product, nicotinamide. The inventors quantified the activity of Nampt in the same lysates harvested for SIRT1 expression and activity. This established that the decline in SIRT1 activity coincided with a similarly abrupt decline in the activity of Nampt (FIG. 22).

To determine if SIRT1 activity was dependent on Nampt activity, the inventors suppressed Nampt activity to 0.11±0.05 of basal level (p<0.001) by incubating SMCs with 10 nM FK866. This had no effect on SIRT1 expression but resulted in substantial loss of SIRT1 activity (FIG. 23). These findings establish that SIRT1 activity depends on Nampt activity, which itself is a vulnerable enzyme as SMCs approach replicative senescence.

Pronounced Extention of Human SMC Longevity by Paired Overexpression of Nampt and SIRT1. In view of these findings, the inventors determined whether overexpressing both SIRT1 and Nampt impacted SMC longevity. As shown in FIG. 24A, the effect was striking. Replicative lifespan of Nampt/SIRT1 SMCs was double that of control SMCs (1.97±0.24-fold increase) and 1.66±0.20-fold that of SMCs overexpressing SIRT1 alone (p<0.001). An inhibitory effect on cell senescence was further revealed by staining cells for senescence-associated beta-galactosidase activity, with significantly fewer senescent SMCs in the double-overexpressing SMC cultures than in either SIRT1-SMC and Nampt-SMC cultures (p<0.05) (FIG. 24B). Double transductants also had significantly higher SIRT1 activity than SMCs expressing SIRT1 alone (FIG. 24C), establishing Nampt as a “feeder” enzyme for SIRT1 activity. These cells also expressed significantly less p21 (FIG. 24D, p<0.05), further supporting the enhanced SIRT1 activity. Interestingly, expression of p16^{INK4A} was not affected by Nampt/SIRT1 overexpression, suggesting that Nampt-SIRT1 axis does not regulate expression of this particular longevity regulator.

Cell Culture. Experiments were performed using primary human SMCs and the HITC6 clonal SMC line, both derived from segments of human internal thoracic artery (Li et al., 1999; Li et al., 2001). SMCs were maintained at 37° C. with 5% CO₂ and cultured in Medium 199 (Invitrogen, Carlsbad, Calif., USA) supplemented with 25 mM HEPES, 10% FBS, 1% penicillin/streptomycin, and 1% L-glutamine. Phoenix cells (distributed by ATCC, Manassas, Va., USA) were maintained at 37° C. with 5% CO₂ and cultured in Dulbecco's modified Eagle's medium (Invitrogen, Carlsbad, Calif., USA). To quantify replication, cells were seeded at a density of 4500 cells/cm2 and population doublings were calculated using the following formula: Log₁₀[# cells harvested]−(Log₁₀ [# cells seeded]/Log 10[2]).

Western Blot Analysis. Protein expression was assessed by Western blot analysis of confluent cultured SMCs, as previously described (Rocnik et al., 2002). Briefly, cells were lysed with RIPA lysis buffer (1% Nonidet P-40, 1% sodium deoxycholate, 0.1% SDS, 150 mM NaCl, 10 mM NaP, 2 mM EDTA, 50 mM NaF) containing protease inhibitor cocktail (Sigma, St. Louis, Mo., USA). Proteins were resolved by SDS-polyacrylamide gel electrophoresis and transferred to Immobilon-P polyvinylidene difluoride membranes (Millipore, Billerica, Mass., USA). Primary antibodies used for Western blot analysis consisted of mouse monoclonal antibodies raised against human p53 (DO-1, Santa Cruz Biotechnology, Santa Cruz, Calif., USA), human p21 (DCS-60, Santa Cruz Biotechnology, Santa Cruz, Calif., USA), and human alpha-tubulin (B512, Sigma, St. Louis, Mo., USA), and rabbit polyclonal antibodies raised against human SIRT1 (ab13749, Abcam, Cambridge, Mass., USA), and human Nampt (BL2122, Bethyl Laboratories, Montgomery, Tex., USA). Secondary antibodies used were horseradish peroxidase conjugated antimouse IgG Fab or anti-rabbit IgG Fab (Amersham Biosciences, Piscataway, N.J., USA). Signal was detected by chemiluminescence (Pierce, Rockford, Ill., USA).

Overexpression of SIRT1 and PBEF in Human SMCs. Stable overexpression of SIRT1 and Nampt in SMCs was carried out using retrovirus-mediated gene delivery, as previously described (Rocnik et al, 2002). Retrovirus containing pQCXIP-Nampt-IRES-PURO or pQCXIP-IRES-PURO (Clontech Laboratories, Mountain View, Calif., USA) was generated by calcium phosphate-mediated transfection of the Phoenix amphotropic retrovirus packaging cell line (ATCC, Manassas, Va., USA). Stable transductants were selected with 3 μg/ml puromycin. SIRT1 was similarly transduced using the pQCXIN retroviral expression vector and selected with 400 μg/ml G418.

Senescence-associated Beta-galactosidase Activity. Senescence-associated beta-galactosidase (SA-β-gal) activity was determined as previously described (Dimri et al, 1995). Briefly, SMCs were fixed in PBS containing 2% formaldehyde/0.2% glutaraldehyde for 3 min at room temperature. Fixed cells were then incubated for 4-6 h at 37° C. with SA-β-gal stain solution containing 150 mM NaCl, 2 mM MgCl, 40 mM citrate, 5 mM potassium ferrocyanide, 5 mM potassium ferricyanide, and 1 mg/ml of 5-bromo-4-chloro-3-indolyl-β-D-galactopyranoside (X-gal) (Invitrogen, Carlsbad, Calif., USA), at pH 6.0. SMCs were counterstained with 2.5 μg/ml Hoechst 33258 to detect cell nuclei. The proportion of SA-β-gal activity positive cells was quantified using light and fluorescence microscopy (Olympus BX51, Center Valley, Pa., USA).

Assessment of SIRT1 Deacetylase Activity. SIRT1 deacetylase activity was quantified in live cells as previously reported (de Boer et al, 2006). SMCs cultured in phenol red-free M199 were incubated without serum for 2 h with 1 μM trichostatin A (Sigma, St. Louis, Mo., USA) followed by addition of 25 μM Fluor-de-Lys-SIRT1 fluorogenic substrate (Biomol, Plymouth Meeting, Pa., USA) for 4 h. Media containing fluorogenic substrate and reaction product was incubated with an equal volume of SIRT1 assay buffer containing 2 mM nicotinamide and developer (Biomol, Plymouth Meeting, Pa., USA). Signal was quantified by spectrofluorometry (Wallac VICTOR-1420, PerkinElmer Life Science, Waltham, Mass., USA) and normalized to total protein content

Assessment of Nicotinamide Phosphoribosyltransferase Activity. Nampt activity was determined in SMC lysates, as previously described (Rongvaux et al, 2002). Briefly, SMCs were lysed in buffer containing 10 mM NaH₂PO₄/Na₂HPO₄, at pH 8.8. Cell lysates were incubated with 0.5 μM [carbonyl-¹⁴C]nicotinamide and 0.5 mM phosphoribosylpyrophosphate for 4 h on a rotating rocker at 37° C. Labeled acetone-precipitable nicotinamide mononucleotide was quantified by scintillation counting (Wallac Trilax 1450 MicroBeta counter, PerkinElmer Life Science, Waltham, Mass., USA).

Quantitative Real-time RT-PCR. SMCs were lysed with Trizol reagent (Invitrogen, Carlsbad, Calif., USA) and RNA extracted by addition of RNase-free chloroform. RNA was precipitated by addition of 70% ethanol and purified using the RNeasy Mini protocol (QIAGEN, Valencia, Calif., USA). Transcript abundance was quantified by real-time RT-PCR. A Taqman-based primer/probe set (Applied Biosystems, Foster City, Calif., USA) was used to detect human p21 (Hs00355782 ml) and a custom designed primer/probe set for p16 was constructed using the exon 1-2 boundary sequence. Values were normalized to the endogenous reference GAPDH (Hs00266705_g1).

Data Analysis. Values are expressed as mean±SD. Statistical analyses were performed using GraphPad Prism software (version 4.0, GraphPad Software Inc., San Diego, Calif., USA). Population doubling plots were fit using non-linear regression. Comparisons were made by Student's t-test on data involving two groups, by one-way analysis of variance (ANOVA) with Tukey post hoc test on experiments involving three or more groups, and by two-way ANOVA for population doubling curves.

### EXAMPLE 4

### Synergy of Nampt and SIRT1 Elevation During Glucose Stress Supports Longevity and Genome Stability

Endothelial dysfunction is a hallmark of Type 2 Diabetes Mellitus (“T2DM”), and is a major factor in the development of cardiovascular complications, including atherosclerosis, hypertension and cardiac and peripheral ischemia. Hyperglycemia can promote endothelial dysfunction during Type II Diabetes. Increased delivery of glucose to the vessel wall can trigger endothelial cell dysfunction and death through several mechanisms, including increased generation and accumulation of reactive oxygen species. The capacity of endothelial cells to withstand glucose overload may depend on the maintenance of glycolysis, glucose oxidation and DNA repair, processes which require a continual supply of NAD+. In mammalian cells, NAD+ is provided by the rate-limiting enzyme, Nampt, through the NAD+ salvage pathway. Emerging evidence suggests that the efficiency of NAD+ synthesis may be a major determinant of cell survival. The inventors have established that overexpression of human Nampt confers resistance to oxidative stress in human vascular smooth muscle cells (SMCs). Since T2DM is associated with glucose-induced oxidative stress in the endothelium, the inventors have determined that Nampt plays a critical role in maintaining endothelial cell viability in response to stressors present in the vasculature during T2DM. Furthermore, since mild oxidative stress can increase SIRT1 expression, and optimal SIRT1 activity is attained only in the presence of sufficient NAD+, the inventors have shown that the protection of endothelial cells from glucose-related stress and aging may require a synergy of Nampt and SIRT1 activity.

In human aortic endothelial cells, prolonged cell culture and subconfluent passage is a model of cell senescence. Cell senescence is associated with a marked decrease in the levels (indicated by immunoblotting) and activity of NamPT (indicated by decreased conversion of NAD+ to B-NMN−) (FIG. 25). To investigate whether natural the decline in NamPT activity induced by aging might affect longevity of these cells over time and in response to glucose stress (such as hyperglycemia—often associated with diabetes, and often leading to cardiovascular complications including atherosclerosis and hypertension), human aortic endothelial cells were transduced with retrovirus encoding either NamPT transgene or eGFP (control). Cells were then passaged to induce senescence under conditions of standard glucose (5 mM glucose) or glucose stress conditions (30 mM glucose) to simulate conditions related to normal cardiovascular aging and that augmented diabetic or other hyperglycemia conditions. Control cells cultured under standard and high glucose conditions demonstrate typical patterns of longevity (FIG. 26). Under normal glucose conditions, cells overexpressing NamPT demonstrate mildly improved longevity (FIG. 26). Whereas under glucose stress conditions, cells overexpressing NamPT demonstrate significantly augmented longevity. This indicates that NamPT overexpression supports longevity in aortic endothelial cells under conditions of glucose stress but not necessarily under normal culture conditions. In addition, senescence is accompanied by typical age- and glucose-induced patterns of tetraploidy (FIG. 27). Nampt overexpressing cells demonstrate no occurrence of age- or high glucose-induced tetraploidy, indicating a protection from genomic instability associated with senescence and glucose overload. Young and old aortic endothelial cells both express elevated levels of endogenous SIRT1 in high glucose relative to those maintained under normal glucose conditions (FIG. 28). As NamPT overexpression enhances longevity in senescing aortic endothelial cells under high glucose stress, it may be argued that while both NamPT overexpression and SIRT1 overexpression individually increase longevity to some extent, it is the combined synergistic elevation of both NamPT and Sirt1 activity and/or levels that is required to have the greatest effect upon longevity. This suggests that the protection of endothelial cells from glucose related stress requires a synergy of NamPT and Sirt1 activity that may either be clinically obtained by augmenting NamPT activity alone and relying upon the stress related elevation of Sirt1, or through the balanced augmentation of both NamPT and Sirt1 levels and/or activity.

Cell Culture. Human aortic endothelial cells (HAEC) purchased from Lonza Corporation were cultured according to protocols provided by the supplier.

Generation of Nampt-overexpressing HAEC populations. HAEC were infected with retrovirus encoding an eGFP-Nampt fusion protein. A control population was infected with eGFP alone. Transduced HAEC were FACS selected for low (eGFP-Namptlo) and high (eGFP-Nampthi) levels of transgene expression based on GFP fluorescence. Control cells were selected to achieve eGFP expression (eGFP+) at a level similar to the eGFP-Namptlo cells. Levels of GFP fluorescence were confirmed by microscopy.

Determination of Nampt expression and activity. Nampt protein content was assessed by immunoblotting. Nampt activity was assessed by the incorporation of radiolabeled nicotinamide into □-nicotinamide mononucleotide.

Assessment of longevity and genomic stability. Triplicate cell populations were plated at 1000 cells/cm2 and were serially subcultured at 80% confluence until growth curves reached plateau. Population doublings were calculated using the standard formula. At certain time points cells were harvested, fixed, and DNA content was analyzed. Tetraploid cells were counted as a proportion of the total population using ModFit software.

Determination of SIRT1 expression. SIRT1 protein content was assessed by immunoblotting.

What is claimed is:

1. A method for treating diseases or conditions in an animal, said method comprising the step of optimizing the intracellular concentration of PBEF in the cells of at least one target tissue of said animal.

2. The method of claim 1, wherein said optimizing of said concentration of PBEF is effected by increasing the intracellular concentration of said PBEF of said animal a sufficient amount of PBEF.

3. The method of claim 1, wherein said optimizing of said concentration of PBEF is effected by administering to said animal a sufficient amount of PBEF to increase the intracellular concentration of said PBEF.

4. The method of claim 3, wherein said administering of PBEF is by at least one route, and said at least one route is one or more of injection, oral administration, anal or other colonic administration, inhalation, intra-peritoneal administration, topical administration, intra-organ administration, infusion of a target tissue, transdermal and parenteral administration, including intravenous, intraperitoneal, subcutaneous, intramuscular, trans-epithelial, nasal, intrapulmonary, intrathecal, rectal and topical modes of administration.

5. The method of claim 3, wherein said administering of PBEF is by at least one route, and said at least one route is one or more of the methods of gene therapy, including the use of one or more viral vectors.

6. The method of claim 3, wherein said one or more viral vectors are chosen from the group comprising adenoviruses, lentiviruses, adeno-associated viruses and non viral plasmid vectors.

7. The method of claim 3, wherein said increasing of said PBEF is effected by promoting the endogenous production of PBEF in the cells of at least one target tissue of said animal.

8. The method of claim 7, wherein said promotion of intracellular production of PBEF is effected by up-regulating the nucleic acid processes which support the production of PBEF.

9. The method of claim 7, wherein said promotion of intracellular production of PBEF is effected by up-regulating the nucleic acid processes which increase the endogenous production of PBEF.

10. The method of claim 7, wherein said promotion of intracellular production of PBEF is effected by down-regulating the nucleic acid processes which repress the production of PBEF.

11. The method of claim 1, wherein said optimization of PBEF is effected by increasing the intracellular concentration of at least one modulator of PBEF.

12. The method of claim 11, wherein said optimization of PBEF is effected by administering to said animal an effective amount of said modulator.

13. The method of claim 11, wherein said administering of said modulator is by at least one route, and said at least one route is one or more of injection, oral administration, anal or other colonic administration, inhalation, intra-peritoneal administration, topical administration, intra-organ administration, infusion of a target tissue, transdermal and parenteral administration, including intravenous, intraperitoneal, subcutaneous, intramuscular, trans-epithelial, nasal, intrapulmonary, intrathecal, rectal and topical modes of administration.

14. The method of claim 11, wherein said modulator is PRPP.

15. The method of claim 11, wherein said increase of PBEF is effected by promoting the endogenous production of PRPP in the cells of at least one target tissue of said animal.

16. The method of claim 14, wherein said promotion of intracellular production of PBEF is effected by up-regulating the nucleic acid processes which increase the production of PRPP.

17. The method of claim 14, wherein said promotion of intracellular production of PBEF is effected by down-regulating the nucleic acid processes which repress the production of PRPP.

18. The method of claim 14, wherein PRPP can be given in combination with at least one form of nicotinamide.

19. The method of claim 18, wherein said nicotinamide may be substituted or in the form of one or more of nicotinic acid; nicotinic acid ribonucleotide; nicotinic acid ribonucleotide, reduced form; nicotinamide ribonucleotide; nicotinamide ribonucleotide, reduced form; nicotinic acid adenine dinucleotide; nicotinic acid adenine dinucleotide, reduced form; nicotinamide adenine dinucleotide (NAD); nicotinamide adenine dinucleotide phosphate (NADP); nicotinamide adenine dinucleotide, reduced form (NADH); and nicotinamide adenine dinucleotide phosphate, reduced form (NADPH) and pharmaceutically acceptable salts thereof.

20. The method of claim 1, wherein said disease or condition is a vascular disease of one or more of the heart, blood vessels and other portions of the cardiovascular system

21. The method of claim 1, wherein said disease or condition is one or more of vascular insufficiency, vascular weakness, progeria, premature senescence of one or more tissues, aging, severe stress on one or more tissues, atherosclerosis, arteriolesclerosis and re-vascularization of injured or weakened tissues or organs.

22. The method of claim 1, wherein said severe stress on one or more tissue is due to one or more of injury, malnutrition, disease, toxic shock and exposure.

23. The method of claim 1, wherein said optimization of PBEF is effected by increasing the intracellular concentration of at least one precursor of PBEF.

24. The method of claim 23, wherein said increase of PBEF is effected by administering to said animal an effective amount of said precursor.

25. The method of claim 23, wherein said administering of said precursor is by at least one route, and said at least one route is one or more of injection, oral administration, anal or other colonic administration, inhalation, intra-peritoneal administration, topical administration, intra-organ administration, infusion of a target tissue, transdermal and parenteral administration, including intravenous, intraperitoneal, subcutaneous, intramuscular, trans-epithelial, nasal, intrapulmonary, intrathecal, rectal and topical modes of administration.

26. The method of claim 23, wherein said precursor is at least one form of nicotinamide.

27. The method of claim 26, wherein said nicotinamide may be substituted or in the form of one or more of nicotinic acid; nicotinic acid ribonucleotide; nicotinic acid ribonucleotide, reduced form; nicotinamide ribonucleotide; nicotinamide ribonucleotide, reduced form; nicotinic acid adenine dinucleotide; nicotinic acid adenine dinucleotide, reduced form; nicotinamide adenine dinucleotide (NAD); nicotinamide adenine dinucleotide phosphate (NADP); nicotinamide adenine dinucleotide, reduced form (NADH); and nicotinamide adenine dinucleotide phosphate, reduced form (NADPH) and pharmaceutically acceptable salts thereof.

28. The method of claim 1 wherein said animal is a human.

29. A composition for optimizing the intracellular concentration of NAD, said composition comprising an effective amount of PBEF.

30. The composition of claim 29, further comprising an effective amount of PRPP.

31. The composition of claim 30, further comprising an effective amount of nicotinamide.

32. The composition of claim 29, further comprising an effective amount of nicotinamide.

33. The composition of claim 32, wherein said nicotinamide may be substituted or in the form of one or more of nicotinic acid; nicotinic acid ribonucleotide; nicotinic acid ribonucleotide, reduced form; nicotinamide ribonucleotide; nicotinamide ribonucleotide, reduced form; nicotinic acid adenine dinucleotide; nicotinic acid adenine dinucleotide, reduced form; nicotinamide adenine dinucleotide (NAD); nicotinamide adenine dinucleotide phosphate (NADP); nicotinamide adenine dinucleotide, reduced form (NADH); and nicotinamide adenine dinucleotide phosphate, reduced form (NADPH) and pharmaceutically acceptable salts thereof.

34. The composition of claim 30, further comprising one or more of an effective amount of a pharmaceutically effective vehicle, a pharmaceutically effective diluent, a pharmaceutically effective cream, a pharmaceutically effective excipient, one or more pharmaceutically effective micelles, a pharmaceutically effective carrier, pharmaceutically acceptable concentrations of salt, buffering agents, preservatives and various compatible carriers.

35. The composition of claim 30, wherein said composition is adaptable for administration by at least one route, and said at least one route is one or more of injection, oral administration, anal or other colonic administration, inhalation, intraperitoneal administration, topical administration, intra-organ administration, infusion of a target tissue, transdermal and parenteral administration, including intravenous, intraperitoneal, subcutaneous, intramuscular, trans-epithelial, nasal, intrapulmonary, intrathecal, rectal and topical modes of administration.

36. The composition of claim 29, wherein said composition is provided in the form of one or more of ingestible tablets, buccal tablets, troches, capsules, elixirs, suspensions, micelle encapsulations, syrups, wafers and the like, or enclosed or enclosable within hard or soft shell gelatin capsules.

37. The composition of claim 29, further comprising one or more of an effective amount of a cosmetically effective vehicle, a cosmetically effective diluent, a cosmetically effective cream, a cosmetically effective excipient, one or more cosmetically effective micelles, a cosmetically effective carrier, cosmetically acceptable concentrations of salt, buffering agents, preservatives and various cosmetically compatible carriers.

38. A composition for optimizing the intracellular concentration of NAD, said composition comprising an effective amount of PRPP.

39. The composition of claim 38, further comprising an effective amount of PBEF.

40. The composition of claim 39, further comprising an effective amount of nicotinamide.

41. The composition of claim 38, further comprising an effective amount of nicotinamide.

42. The composition of claim 41, wherein said nicotinamide may be substituted or in the form of one or more of nicotinic acid; nicotinic acid ribonucleotide; nicotinic acid ribonucleotide, reduced form; nicotinamide ribonucleotide; nicotinamide ribonucleotide, reduced form; nicotinic acid adenine dinucleotide; nicotinic acid adenine dinucleotide, reduced form; nicotinamide adenine dinucleotide (NAD); nicotinamide adenine dinucleotide phosphate (NADP); nicotinamide adenine dinucleotide, reduced form (NADH); and nicotinamide adenine dinucleotide phosphate, reduced form (NADPH) and pharmaceutically acceptable salts thereof.

43. The composition of claim 39, further comprising one or more of an effective amount of a pharmaceutically effective vehicle, a pharmaceutically effective diluent, a pharmaceutically effective cream, a pharmaceutically effective excipient, one or more pharmaceutically effective micelles, a pharmaceutically effective carrier, pharmaceutically acceptable concentrations of salt, buffering agents, preservatives and various compatible carriers.

44. The composition of claim 39, wherein said composition is adaptable for administration by at least one route, and said at least one route is one or more of injection, oral administration, anal or other colonic administration, inhalation, intraperitoneal administration, topical administration, intra-organ administration, infusion of a target tissue, transdermal and parenteral administration, including intravenous, intraperitoneal, subcutaneous, intramuscular, trans-epithelial, nasal, intrapulmonary, intrathecal, rectal and topical modes of administration.

45. The composition of claim 38, wherein said composition is provided in the form of one or more of ingestible tablets, buccal tablets, troches, capsules, elixirs, suspensions, micelle encapsulations, syrups, wafers and the like, or enclosed or enclosable within hard or soft shell gelatin capsules.

46. The composition of claim 38, further comprising one or more of an effective amount of a cosmetically effective vehicle, a cosmetically effective diluent, a cosmetically effective cream, a cosmetically effective excipient, one or more cosmetically effective micelles, a cosmetically effective carrier, cosmetically acceptable concentrations of salt, buffering agents, preservatives and various cosmetically compatible carriers.


SEQUENCE LISTING (6 sequences)

SEQ ID NO: 1 - DNA, 19 bp, Homo sapiens
ggaaggtgaaatatgagga

SEQ ID NO: 2 - DNA, 19 bp, Homo sapiens
atgttctcttcacggtgga

SEQ ID NO: 3 - DNA, 19 bp, Homo sapiens
agggccgattatctttaca

SEQ ID NO: 4 - DNA, 15 bp, Homo sapiens
cagttgctgatccca

SEQ ID NO: 5 - DNA, 23 bp, Homo sapiens
tgcagctatgttgtaaccaatgg

SEQ ID NO: 6 - DNA, 21 bp, Homo sapiens
acaaaaggtcgaaaaagggcc